(19) United States
(12) Patent Application Publication
(10) Pub. No.: US 2016/0311921 A1
(43) Pub. Date: Oct. 27, 2016

(54) METHODS FOR TREATMENT OF OVARIAN CANCER

(71) Applicant: MORPHOTEK ,INC., Exton, PA (US)

(72) Inventors: Charles SCHWEIZER, Doylestown, PA (US); Daniel John O'SHANNESSY, Schwenksville, PA (US)

(73) Assignee: Morphotek ,Inc.

(21) Appl. No.: 14/898,905

(22) PCT Filed: Jun. 20, 2014
(86) PCT No.: PCT/US2014/043402

§ 371 Date: Dec. 16, 2015

Related U.S. Application Data
(60) Provisional application No. 61/837,543, filed on Jun. 20, 2013.

(51) Int. Cl. C07K 16/30; G01N 33/574; A61K 9/00; A61K 45/06; A61K 49/00; A61K 39/395

(52) U.S. Cl. CPC C07K 16/3092; A61K 49/00; A61K 39/39558; A61K 9/0019; A61K 45/06; G01N 33/57449; A61K 2039/505

(57) ABSTRACT

Provided herein are methods of identifying a subpopulation of ovarian cancer patients who would be responsive to treatment regimens that target folate receptor alpha (FRA)-expressing ovarian tumors and methods of treatment of such patients using an anti-FRA therapeutic agent, such as an antigen-binding protein (e.g., antibody or antigen-binding fragment thereof) that specifically binds to FRA. Also provided are related kits for identification and treatment of the subpopulation of ovarian cancer patients.

### CROSS-REFERENCE TO RELATED APPLICATIONS

This application claims the benefit of U.S. provisional application 61/837,543, filed Jun. 20, 2013, which is incorporated herein by reference.

### TECHNICAL FIELD

The subject matter described herein relates to methods of identifying and methods of treating a subpopulation of ovarian cancer patients who would be responsive to treatment regimens that target folate receptor alpha (FRA)-expressing ovarian tumors and treatment of such patients using an anti-FRA therapeutic agent.

### BACKGROUND

According to the National Cancer Institute, an estimated 22,240 new cases of ovarian cancer will be diagnosed in the United States in 2013. In addition, an estimated 14,030 deaths from ovarian cancer will occur in the United States in 2013. Ovarian cancer is considered a “silent killer” because of the absence of specific symptoms until late in the disease when 75% of the cases are diagnosed, five year survival rates are less than 30%, and a 70% recurrence rate is expected. [O'Shannessy et al., Journal of Ovarian Research 2013, 6:29].

Folate receptor alpha (FRA) is a glycosylphosphatidyl-inositol-linked protein that is overexpressed in several epithelial malignancies, including ovarian, renal, lung, and breast cancers [Elnakat and Ratnam, Front Biosci. 2006; 11:506-19]. FRA is an attractive candidate for targeted biologic therapy of ovarian cancer [Reddy, et al., Curr Pharm Biotechnol. 2005; 6:131-50]. It is reported to be expressed in the majority of non-mucinous epithelial ovarian tumors at levels 10- to 100-fold higher than its normal expression in the kidney and on lung and breast epithelial cells [Parker, et al., Anal Biochem. 2005; 338:284-93]. In addition, FRA is a tumor antigen, with 70% of women with ovarian or breast cancer showing measurable immune responses against this protein [Knutson, et al., J Clin Oncol. 2006; 24:4254-61].

The tumor specificity and high levels of FRA expression in some ovarian cancers have generated significant enthusiasm for testing strategies targeting FRA in ovarian cancer patients. For example, MORAb-003 (USAN:farletuzumab), a humanized, high-affinity monoclonal antibody against FRA is currently undergoing clinical development for treatment of ovarian cancer patients after showing cell-mediated cytotoxicity, complement-dependent killing, and non-immune mediated, FRA-dependent inhibition of growth under folate-limiting conditions [Ebel, et al. Cancer Immun. 2007; 7:6].

A pressing need exists, however, for methods for identifying ovarian cancer patients who would be responsive to treatment regimens that target folate receptor alpha (FRA)-expressing ovarian tumors. The methods and kits described herein satisfy this need.

### SUMMARY

Provided herein are methods for identifying a subject having a folate receptor alpha (FRA)-expressing ovarian cancer that will be responsive to treatment with an anti-FRA therapeutic agent and methods of treating a subject with folate receptor alpha (FRA)-expressing ovarian cancer. In some embodiments of the described methods, the ovarian cancer is epithclial ovarian cancer. In some embodiments, the ovarian cancer is either platinum-sensitive or platinum-resistant. In some embodiments, the subject received a platinum-based first-line therapy.

In some embodiments of the described methods for identifying a subject having a folate receptor alpha (FRA)-expressing ovarian cancer that will be responsive to treatment with an anti-FRA therapeutic agent, the methods involve determining a baseline level of cancer antigen 125 (CA125) expression in the subject. A baseline CA125 level that is less than about eight times the upper limit of normal (ULN) for CA125, preferably less than about seven times the ULN for CA125, more preferably less than about six times the ULN for CA125, more preferably less than about five times the ULN for CA125, more preferably less than about four times the ULN for CA125, more preferably less than about three times the ULN for CA125, more preferably less than about two times the ULN for CA125 and, in some embodiments, less than about the ULN for CA125 is indicative of a subject who would benefit from treatment with an anti-FRA therapeutic agent. A baseline CA125 level that is less than about 164 units/ml, preferably less than about 150 units/ml, more preferably less than about 140 units/ml, more preferably less than about 130 units/ml, more preferably less than about 120 units/ml, more preferably less than about 110 units/ml, more preferably less than about 100 units/ml, even more preferably less than about 90 units/ml, more preferably less than about 80 units/ml, more preferably less than about 70 units/ml, more preferably less than about 63 units/ml, in some embodiments less than about 42 units/ml, in some embodiments less than about 35 units/ml, and in some embodiments less than about 21 units/ml is indicative of a subject who would benefit from treatment with an anti-FRA therapeutic agent.

In some embodiments of the provided methods of treating a subject with folate receptor alpha (FRA)-expressing ovarian cancer, the baseline level of cancer antigen 125 (CA125) expression of the subject is determined and, when the CA125 level is less than about eight times the upper limit of normal (ULN) for CA125, preferably less than about seven times the ULN for CA125, more preferably less than about six times the ULN for CA125, more preferably less than about five times the ULN for CA125, more preferably less than about four times the ULN for CA125, more preferably less than about three times the ULN for CA125, more preferably less than about two times the ULN for CA125 and, in some embodiments, less than about the ULN for CA125, a therapeutically effective amount of an anti-FRA therapeutic agent is administered to the subject. In some embodiments of the provided methods of treating a subject with folate receptor alpha (FRA)-expressing ovarian cancer, the baseline level of cancer antigen 125 (CA125) expression of the subject is determined and, when the CA125 level is less than about 164 units/ml, preferably less than about 150 units/ml, more preferably less than about 140 units/ml, more preferably less than about 130 units/ml, more preferably less than about 120 units/ml, more preferably less than about 110 units/ml, more preferably less than about 100 units/ml, even more preferably less than about 90 units/ml, more preferably less than about 80 units/ml, more preferably less than about 70 units/ml, more preferably less than about 63 units/ml, in some embodiments less than about 42 units/ml, in some embodiments less than about 35 units/ml, and in some embodiments less than about 21 units/ml, a therapeutically effective amount of an anti-FRA therapeutic agent is administered to the subject.

In accordance with the methods described herein, the baseline CA125 level may be determined ex vivo or in vivo (e.g., in a biological sample obtained from the subject).

In some embodiments of the methods described herein, the anti-FRA therapeutic agent is an antigen-binding protein that specifically binds FRA, such as an antibody that specifically binds FRA or an antigen-binding fragment of such antibody. In preferred embodiments, the anti-FRA therapeutic agent is farletuzumab.

In some embodiments of the methods for identifying a subject having a folate receptor alpha (FRA)-expressing ovarian cancer that will be responsive to treatment with an anti-FRA therapeutic agent and methods of treating a subject with folate receptor alpha (FRA)-expressing ovarian cancer provided herein, the methods further involve a determination of a FRA concentration of the subject and comparison of the FRA level of the subject to the level of FRA in a control sample, wherein an increase in the level of FRA in the sample derived from the subject as compared to the level of FRA in the control sample is indicative that the subject would benefit from treatment with an anti-FRA therapeutic agent. The level of FRA may be either a measurement of the FRA level in the subject at a single timepoint or may involve measurement of FRA levels in the subject at at least two points in time. Determination of the baseline level of FRA in the subject may be performed upon diagnosis, upon surgical resection, upon initiation of first-line therapy, upon completion of first-line therapy, upon symptomatic progression, serologic progression, and/or radiologic progression of the cancer, upon initiation of second-line therapy, and/or upon completion of second-line therapy.

In some embodiments of the methods for identifying a subject having a folate receptor alpha (FRA)-expressing ovarian cancer that will be responsive to treatment with an anti-FRA therapeutic agent and methods of treating a subject with folate receptor alpha (FRA)-expressing ovarian cancer provided herein, the methods further involve a determination of a baseline serum albumin concentration of the subject. A baseline serum albumin (SA) concentration of at least 3.2 g/dL is further indicative of a subject who would benefit from treatment with an anti-FRA therapeutic agent. The baseline level of SA may be either a measurement of the SA level in the subject at a single timepoint or may involve measurement of SA levels in the subject at at least two points in time. Determination of the baseline level of SA in the subject may be performed upon diagnosis, upon surgical resection, upon initiation of first-line therapy, upon completion of first-line therapy, upon symptomatic progression, serologic progression, and/or radiologic progression of the cancer, upon initiation of second-line therapy, and/or upon completion of second-line therapy.

In some embodiments of the methods of treatment provided herein, serum anti-FRA therapeutic agent concentration of the subject is determined. A minimum serum concentration of at least about 57.6 μg/ml, more preferably at least about 88.8 μg/ml, is indicative of a positive therapeutic response to the anti-FRA therapeutic agent.

In some embodiments of the described methods of treatment, the anti-FRA therapeutic agent is administered to the subject to achieve a minimum serum concentration. In preferred embodiments, the minimum serum concentration achieved is at least about 57.6 μg/ml, more preferably at least about 88.8 μg/ml, within about three weeks, preferably within about two weeks, and more preferably within about one week of administration of the initial dose of the anti-FRA therapeutic agent to the subject. In preferred embodiments, once such minimum serum concentration is achieved in a subject, the subject's serum level of the anti-FRA therapeutic agent remains above the Cmin or Ctrough for the remainder of therapy with the anti-FRA therapeutic agent.

In some embodiments of the methods of treatment provided herein, the anti-FRA therapeutic agent average area under the curve (AUC) pharmacokinetic (PK) exposure level is determined. For example, when the anti-FRA therapeutic agent is farletuzumab, farletuzumab average AUC PK exposure level is determined. An anti-FRA therapeutic agent average AUC PK exposure level of about 15.22 mg·h/ml or more, more preferably at least about 22.2 mg·h/L, is indicative of a positive therapeutic response to the anti-FRA therapeutic agent.

Some embodiments of the methods of treatment provided herein further involve administration of a therapeutically effective amount of a platinum-containing compound and/or a taxane to the subject in addition to the anti-FRA therapeutic agent Exemplary platinum-containing compounds are cisplatin or carboplatin. Examples of taxanes for use in the methods of treatment include but are not limited to paclitaxel, docetaxel, and semi-synthetic, synthetic, and/or modified versions and formulations thereof, including but not limited to nab-paclitaxel (Abraxane®), cabazitaxel (Jevtana®), DJ-927 (Tesetaxel®), paclitaxel poliglumex (Opaxio®), XRP9881 (Larotaxel®), EndoTAG+paclitaxel (EndoTAG®-1), Polymeric-micellar paclitaxel (Genexol-PM®), DHA-paclitaxel (Taxprexin®)), and BMS-184476

In some embodiments of the methods described herein, the subject may have received surgical resection of the ovarian cancer, first-line platinum-based therapy, first-line taxane-based therapy, and/or first-line platinum- and taxane-based therapy for treatment of the ovarian cancer prior to determining the baseline level of CA125. In some embodiments of the methods described herein in which the subject received surgical resection of the ovarian cancer, first-line platinum-based therapy, first-line taxane-based therapy, and/or first-line platinum and taxane-based therapy for treatment of the ovarian cancer prior to determining the baseline level of CA125, the subject may have exhibited symptomatic progression, serologic progression, and/or radiologic progression of the ovarian cancer prior to the step of determining the baseline level of CA125.

Further provided herein are kits for identifying a subject having ovarian cancer that will be responsive to treatment with an anti-folate receptor alpha (FRA) therapeutic agent. In some embodiments, the kits contain an anti-CA125 antibody, a vessel for containing the antibody when not in use, and instructions for using the anti-CA125 antibody for determining the level of CA125 of a subject. The instructions may specify that a baseline CA125 level is less than about eight times the upper limit of normal (ULN) for CA125, preferably less than about seven times the ULN for CA125, more preferably less than about six times the ULN for CA125, more preferably less than about five times the ULN for CA125, more preferably less than about four times the ULN for CA125, more preferably less than about three times the ULN for CA125, more preferably less than about two times the ULN for CA125 and, in some embodiments, less than about the ULN for CA125, is indicative of a subject who would benefit from treatment with an anti-FRA therapeutic agent. Alternatively, the instructions may specify that a baseline CA125 level that is less than about 164 units/ml, preferably less than about 150 units/ml, more preferably less than about 140 units/ml, more preferably less than about 130 units/ml, more preferably less than about 120 units/ml, more preferably less than about 110 units/ml, more preferably less than about 100 units/ml, even more preferably less than about 90 units/ml, more preferably less than about 80 units/ml, more preferably less than about 70 units/ml, more preferably less than about 63 units/ml, in some embodiments less than about 42 units/ml, in some embodiments less than about 35 units/ml, and in some embodiments less than about 21 units/ml, is indicative of a subject who would benefit from treatment with an anti-FRA therapeutic agent. In some embodiments, the kits also contain an anti-FRA antibody, a vessel for containing the anti-FRA antibody when not in use, and instructions for using the anti-FRA antibody for determining the level of FRA of a subject. In some embodiments, the kits may contain an anti-serum albumin (SA) antibody, a vessel for containing the anti-SA antibody when not in use, and instructions for using the anti-SA antibody for determining the level of SA of a subject.

Also provided herein are kits for treating a subject having ovarian cancer that will be responsive to treatment with an anti-FRA therapeutic agent comprising the anti-FRA therapeutic agent, a vessel for containing the anti-FRA therapeutic agent when not in use, and instructions for use of the anti-FRA therapeutic agent. The instructions may specify that a baseline CA125 level is less than about eight times the upper limit of normal (ULN) for CA125, preferably less than about seven times the ULN for CA125, more preferably less than about six times the ULN for CA125, more preferably less than about five times the ULN for CA125, more preferably less than about four times the ULN for CA125, more preferably less than about three times the ULN for CA125, more preferably less than about two times the ULN for CA125 and, in some embodiments, less than about the ULN for CA125, is indicative of a subject who would benefit from treatment with an anti-FRA therapeutic agent. Alternatively, the instructions may specify that a baseline CA125 level that is less than about 164 units/ml, preferably less than about 150 units/ml, more preferably less than about 140 units/ml, more preferably less than about 130 units/ml, more preferably less than about 120 units/ml, more preferably less than about 110 units/ml, more preferably less than about 100 units/ml, even more preferably less than about 90 units/ml, more preferably less than about 80 units/ml, more preferably less than about 70 units/ml, more preferably less than about 63 units/ml, in some embodiments less than about 42 units/ml, in some embodiments less than about 35 units/ml, and in some embodiments less than about 21 units/ml, is indicative of a subject who would benefit from treatment with the anti-FRA therapeutic agent. Farletuzumab is the preferred anti-FRA therapeutic agent for inclusion in the kits. In some embodiments, the kits for treating a subject having ovarian cancer that will be responsive to treatment with an anti-FRA therapeutic agent also contain an anti-CA125 antibody, a vessel for containing the anti-CA125 antibody when not in use, and instructions for using the anti-CA125 antibody for determining a baseline level of CA125 of a subject.

Additional aspects of the summarized subject matter are provided in greater detail in the detailed description and provided examples and associated figures.

### BRIEF DESCRIPTION OF THE DRAWINGS

FIG. 1 shows CA125 effect on median progression-free survival (PFS) of patients having a baseline CA125 serum concentration three times ULN (3×ULN=63 U/ml) or less. As part of the primary analysis, efficacy of farletuzumab was assessed based on the biomarker CA125 to identify efficacy within a subgroup of patients above or below a defined threshold of three times the upper limit of normal for CA125. Kaplan-Meier curves of patients exhibiting three times or less the ULN of CA125 values treated with 1.25 mg/kg FAR+carboplatin/Taxane; 2.5 mg/kg FAR+carboplatin/taxane; and placebo+carboplatin/taxane are plotted for the primary Intent to Treat population (FIT). In this biomarker subgroup, patients receiving the high dose of farletuzumab (2.5 mg/kg) has a statistically significant difference in median PFS of 13.6 months compared to 8.8 months in placebo (HR=0.49; p=0.0014). Solid line/open circle represents results for the group that received placebo+carboplatin/Taxane. Dotted line, closed circle represents results for treatment group that received 1.25 mg/kg FAR+Carboplatin/Taxane. Dotted line, X represents results for the treatment group that received 2.5 mg/kg FAR+Carboplatin/Taxane.

FIG. 2 shows CA125 effect on median progression-free survival (PFS) of patients having a baseline CA125 serum concentration greater than three times ULN (63 U/ml). Kaplan-Meier curves of patients exhibiting greater than three times the ULN of CA125 values treated with 1.25 mg/kg FAR+carboplatin/Taxane (low dose of farletuzumab); 2.5 mg/kg FAR+carboplatin/taxane (high dose of farletuzumab); and placebo+carboplatin/taxane are plotted for the primary Intent to Treat population (ITT). Median PFS was 9 months in placebo and 8.8 months in both farletuzumab low and high doses. Therefore, farletuzumab did not appear to have a positive effect on PFS based on a patient subgroup with higher levels of CA125.

FIG. 3 shows a Kaplan-Meier curve comparing PFS in placebo patients by baseline 3×ULN CA125 level. 93 of 357 total placebo patients had a CA125<3×ULN, with a median PFS of 8.8 months compared to 9.0 months in the >3×ULN patients. The median PFS is similar and there is not a statistically significant difference between the two groups (HR=0.88; p=0.48). Therefore, baseline CA125 in patients who received placebo combined with standard of care chemotherapy did not have any statistical or clinical difference in median PFS, where CA125 did not indicate any prognostic or predictive effect in this patient population.

FIG. 4 illustrates the dose-dependent inhibition of farletuzumab cytotoxicity by CA125. Antibodies (Farletuzumab or negative control IgG), effector cells, and increasing concentrations of CA125 were added to human FRA-expressing Chinese hamster ovary (CHO-hFRA) target cells. Increasing luminescence indicates effector cell activation (ADCC activity) as described by Promega ADCC Reporter Bioassay Core Kit. As shown in FIG. 4, there was a dose-dependent inhibition of Farletuzumab ADCC activity with increasing levels of CA125, with a maximal inhibition of approximately 50%.

FIG. 5 illustrates the optimization of clinical effects of farletuzumab as measured by progression-free survival (PFS) versus CA125 levels. A threshold of three times the CA125 ULN was prespecified in analysis plans to identify differences between levels of elevated CA125, and demonstrated a positive effect for the lower CA125 subgroup. Accordingly, additional analysis has demonstrated additional potential cutpoint values that could be used to optimize a CA125 value cutpoint that maximize the treatment effect in the largest subgroup possible. FIG. 5 graphs hazard ratios for CA125 at CA125 cutpoint values from 0-250 in patients with high median pharmacokinetic (PK) exposure levels independent of farletuzumab dose. The lower curve (blue circles) indicates hazard ratios for subjects at or below the CA125 value for that estimate, while the higher curve (red crosses) illustrates the hazard ratios for those subjects above that same cutpoint. As shown, a robust clinical effect is observed in patients with high farletuzumab PK exposure levels exhibiting about 130 U/ml or less of CA125, with a hazard ratio of approximately 0.5 or better up to this value.

FIG. 6 illustrates median progression-free survival (PFS) for patients based on Cmin farletuzumab pharmacokinetic exposure levels. Kaplan Meier curves for PFS were developed demonstrating a difference in PFS by median average Cmin or lowest point PK trough levels independent of the assigned farletuzumab dose. PFS in subjects with farletuzumab Cmin concentrations above median levels (>57.6 μg/mL) showed a statistically significant improvement in PFS when compared to placebo (p=0.002, HR=0.679, 95% CI [0.553-0.832]). Patients in the higher average farletuzumab Cmin had an average PFS of 10.3 months (higher plotted curve). Patients with a higher average farletuzumab Cmin level had better PFS than those patients with placebo and lower average Cmin, indicating an exposure response relationship.

FIG. 7 illustrates progression-free survival by quartile of farletuzumab average area under the curve (AUC) pharmacokinetic exposure levels. Kaplan-Meier plots for subjects with farletuzumab average AUC pharmacokinetic exposure levels above median levels (>15.22 mg·h/mL) and in particular for the upper quartile (Q4>22.8 mg·h/mL) showed a significant relationship for PFS in comparison to placebo (p=0.001, HR=0.641, 95% CI [0.491-0.836]). PFS for those subjects with farletuzumab in Q4 (>22.2 mg·h/L) had a longer PFS when compared to other lower AUC quartiles, and the overall Q4 PFS was 10.3 months compared to 8.84 months in placebo.

FIG. 8 shows PFS vs above & below Median CA125 (IU/mL) combined with Q4 farletuzumab AUC. This figure plots a Kaplan-Meier curve for PFS comparing median CA125 levels and placebo in the farletuzumab highest concentration population. Patients in the highest 75% quartile concentration level by AUC (Q4) are divided above or below the median CA125 value (164 IU/ml). Those Q4 AUC concentration patients with a CA125 below the median have a statistically significant difference in PFS of 12.5 months versus 8.84 in placebo (HR=0.46; p=0.000094). Patients with this same higher Q4 AUC level that have a higher than median CA125 only have an improvement of PFS of 9.46 months which is not statistically significant.

FIG. 9 illustrates the relationship between farletuzumab exposure and patient albumin levels. In the population pharmacokinetic analysis, farletuzumab clearance was identified to decline with increasing baseline albumin levels. Lower baseline albumin is associated with a decrease in farletuzumab dose-normalized concentration exposure (AUC) levels.

FIG. 10 illustrates simulated weekly farletuzumab concentration-time profiles following administration of farletuzumab. Modeling has been used to compare farletuzumab concentration levels based on increasing weekly doses. Results of the exposure PFS analysis indicate that a median farletuzumab Cmin (or Ctrough) level of 57.6 μg/mL can correlate with an improvement of PFS, which is indicated in the lower dotted horizontal line. Weekly doses of 2.5 mg/kg have a 71% attainment rate to reach the median Ctrough level and a 28% attainment rate to reach a higher Q4 Ctrough level. The model indicates that a minimum dose of 5 mg/kg weekly is necessary to reach a 99% attainment rate for median Ctrough level and 89% attainment rate for the Q4 Ctrough target.

FIG. 11 illustrates simulated farletuzumab concentration-time profiles following weekly and loading dose administration of farletuzumab. Modeling has been used to compare farletuzumab concentration levels based on higher weekly doses and an initial loading dose to reach target concentration levels faster. Results of the exposure PFS analysis indicate that a median Cmin (or Ctrough) level of 57.6 μg/mL correlates with an improvement of PFS, which is indicated in the lower dotted horizontal line. The model indicates that a minimum dose of 5 mg/kg farletuzumab weekly is necessary to reach a 99% attainment rate for median Ctrough, and the use of a 10 mg/kg farletuzumab loading dose demonstrates more rapid attainment of the target Ctrough level of both the median and Q4 level.

### DETAILED DESCRIPTION OF ILLUSTRATIVE EMBODIMENTS

Various terms relating to aspects of the description are used throughout the specification and claims. Such terms are to be given their ordinary meaning in the art unless otherwise indicated. Other specifically defined terms are to be construed in a manner consistent with the definitions provided herein.

As used in this specification and the appended claims, the singular forms “a,” “an,” and “the” include plural referents unless the content clearly dictates otherwise. Thus, for example, reference to “a cell” includes a combination of two or more cells, and the like.

The term “about” as used herein when referring to a measurable value such as an amount, a temporal duration, and the like, is meant to encompass variations of up to ±10% from the specified value, as such variations are appropriate to perform the disclosed methods. Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

The term “antibody” refers to (a) immunoglobulin polypeptides, i.e., polypeptides of the immunoglobulin family that contain an antigen binding site that specifically binds to a specific antigen (e.g., folate receptor alpha), including all immunoglobulin isotypes (IgG, IgA, IgE, IgM, IgD, and IgY), classes (e.g. IgG1, IgG2, IgG3, IgG4, IgA1, IgA2), subclasses, and various monomeric and polymeric forms of each isotype, unless otherwise specified, and (b) conservatively substituted variants of such immunoglobulin polypeptides that immunospecifically bind to the antigen (e.g., folate receptor alpha). Antibodies are generally described in, for example, Harlow & Lane, Antibodies: A Laboratory Manual (Cold Spring Harbor Laboratory Press, 1988). Unless otherwise apparent from the context, reference to an antibody also includes antibody derivatives as described in more detail below.

“Antibody fragments” comprise a portion of a full length antibody, generally the antigen-binding or variable region thereof, such as Fab, Fab′, F(ab′)₂, and Fv fragments; diabodies; linear antibodies; single-chain antibody molecules; and multispecific antibodies formed from antibody fragments. Various techniques have been developed for the production of antibody fragments, including proteolytic digestion of antibodies and recombinant production in host cells; however, other techniques for the production of antibody fragments will be apparent to the skilled practitioner. In some embodiments, the antibody fragment of choice is a single chain Fv fragment (scFv). “Single-chain Fv” or “scFv” antibody fragments comprise the V_{H} and V_{L} domains of antibody, wherein these domains are present in a single polypeptide chain. Generally, the Fv polypeptide further comprises a polypeptide linker between the V_{H} and V_{L} domains which enables the scFv to form the desired structure for antigen binding. For a review of scFv and other antibody fragments, see James D. Marks, Antibody Engineering, Chapter 2, Uxford University Press (199) (Carl K. Borrebaeck, Ed.).

An “antibody derivative” means an antibody, as defined above, that is modified by covalent attachment of a heterologous molecule such as, e.g., by attachment of a heterologous polypeptide (e.g., a cytotoxin) or therapeutic agent (e.g., a chemotherapeutic agent), or by glycosylation, deglycosylation, acctylation or phosphorylation not normally associated with the antibody, and the like.

The term “monoclonal antibody” refers to an antibody that is derived from a single cell clone, including any eukaryotic or prokaryotic cell clone, or a phage clone, and not the method by which it is produced. Thus, the term “monoclonal antibody” is not limited to antibodies produced through hybridoma technology.

An “antigen” is an entity to which an antibody specifically binds. For example, folate receptor alpha is the antigen to which an anti-folate receptor-alpha antibody specifically binds.

The terms “cancer” and “tumor” are well known in the art and refer to the presence, e.g., in a subject, of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells may exist alone within a subject, or may be non-tumorigenic cancer cells, such as leukemia cells. As used herein, the term “cancer” includes pre-malignant as well as malignant cancers.

As used herein, the term “folate receptor alpha” (also referred to as FRA, FR-alpha, FOLR-1 or FOLR1) refers to the alpha isoform of the high affinity receptor for folate. Membrane bound FRA is attached to the cell surface by a glycosyl phosphatidylinositol (GPI) anchor, recycles between extracellular and endocytic compartments and is capable of transporting folate into the cell. FRA is expressed in a variety of epithelial tissues including those of the female reproductive tract, placenta, breast, kidney proximal tubules, choroid plexus, lung and salivary glands. Soluble forms of FRA may be derived by the action of proteases or phospholipase on membrane anchored folate receptors.

The consensus nucleotide and amino acid sequences for human FRA are set forth herein as SEQ ID NOs: 9 and 10, respectively.

| SEQ ID NO: 9 | |
|---|---|
| tcaaggttaa acgacaagga cagacatggc tcagcggatg acaacacagc tgctgctcct | 60 |
| tctagtgtgg gtggctgtag taggggaggc tcagacaagg attgcatggg ccaggactga | 120 |
| gcttctcaat gtctgcatga acgccaagca ccacaaggaa aagccaggcc ccgaggacaa | 180 |
| gttgcatgag cagtgtcgac cctggaggaa gaatgcctgc tgttctacca acaccagcca | 240 |
| ggaagcccat aaggatgttt cctacctata tagattcaac tggaaccact gtggagagat | 300 |
| ggcacctgcc tgcaaacggc atttcatcca ggacacctgc ctctacgagt gctcccccaa | 360 |
| cttggggccc tggatccagc aggtggatca gagctggcgc aaagagcggg tactgaacgt | 420 |
| gcccctgtgc aaagaggact gtgagcaatg gtgggaagat tgtcgcacct cctacacctg | 480 |
| caagagcaac tggcacaagg gctggaactg gacttcaggg tttaacaagt gcgcagtggg | 540 |
| agctgcctgc caacctttcc atttctactt ccccacaccc actgttctgt gcaatgaaat | 600 |
| ctggactcac tcctacaagg tcagcaacta cagccgaggg agtggccgct gcatccagat | 660 |
| gtggttcgac ccagcccagg gcaaccccaa tgaggaggtg gcgaggttct atgctgcagc | 720 |
| catgagtggg gctgggccct gggcagcctg gcctttcctg cttagcctgg ccctaatgct | 780 |
| gctgtggctg ctcagctgac ctccttttac cttctgatac ctggaaatcc ctgccctgtt | 840 |
| cagccccaca gctcccaact atttggttcc tgctccatgg tcgggcctct gacagccact | 900 |
| ttgaataaac cagacaccgc acatgtgtct tgagaattat ttggaaaaaa aaaaaaaaaa | 960 |
| aa | 962 |

SEQ ID NO: 10
Met Ala Gln Arg Met Thr Thr Gln Leu Leu Leu Leu Leu Val Trp Val
Ala Val Val Gly Glu Ala Gln Thr Arg Ile Ala Trp Ala Arg Thr Glu
Leu Leu Asn Val Cys Met Asn Ala Lys His His Lys Glu Lys Pro Gly
Pro Glu Asp Lys Leu His Glu Gln Cys Arg Pro Trp Arg Lys Asn Ala
Cys Cys Ser Thr Asn Thr Ser Gln Glu Ala His Lys Asp Val Ser Tyr
Leu Tyr Arg Phe Asn Trp Asn His Cys Gly Glu Met Ala Pro Ala Cys
Lys Arg His Phe Ile Gln Asp Thr Cys Leu Tyr Glu Cys Ser Pro Asn
Leu Gly Pro Trp Ile Gln Gln Val Asp Gln Ser Trp Arg Lys Glu Arg
Val Leu Asn Val Pro Leu Cys Lys Glu Asp Cys Glu Gln Trp Trp Glu
Asp Cys Arg Thr Ser Tyr Thr Cys Lys Ser Asn Trp His Lys Gly Trp
Asn Trp Thr Ser Gly Phe Asn Lys Cys Ala Val Gly Ala Ala Cys Gln
Pro Phe His Phe Tyr Phe Pro Thr Pro Thr Val Leu Cys Asn Glu Ile
Trp Thr His Ser Tyr Lys Val Ser Asn Tyr Ser Arg Gly Ser Gly Arg
Cys Ile Gln Met Trp Phe Asp Pro Ala Gln Gly Asn Pro Asn Glu Glu
Val Ala Arg Phe Tyr Ala Ala Ala Met Ser Gly Ala Gly Pro Trp Ala
Ala Trp Pro Phe Leu Leu Ser Leu Ala Leu Met Leu Leu Trp Leu Leu
Ser

Variants, for example, naturally occurring allelic variants or sequences containing at least one amino acid substitution, are encompassed by the terms as used herein.

As used herein, the term “not bound to a cell” refers to a protein that is not attached to the cellular membrane of a cell, such as a cancerous cell. In a particular embodiment, the FRA not bound to a cell is unbound to any cell and is freely floating or solubilized in biological fluids, e.g., urine or serum. For example, a protein that is not bound to a cell may be shed, secreted or exported from normal or cancerous cells, for example, from the surface of cancerous cells, into biological fluids.

The “level” of a specified protein, as used herein, refers to the level of the protein as determined using any method known in the art for the measurement of protein levels. Such methods include, for example, electrophoresis, capillary electrophoresis, high performance liquid chromatography (HPLC), thin layer chromatography (TLC), hyperdiffusion chromatography, fluid or gel precipitation reactions, absorption spectroscopy, colorimetric assays, spectrophotometric assays, flow cytometry, immunodiffusion (single or double), solution phase assay, immunoelectrophoresis, Western blotting, radioimmunoassay (RIA), enzyme-linked immunosorbent assays (ELISAs), immunofluorescent assays, and electrochemiluminescence immunoassay (exemplified below), and the like. In a preferred embodiment, the level is determined using antibody based techniques, as described in more detail herein.

Antibodies used in immunoassays to determine the level of expression of a specified protein, such as for example, CA125 or FRA, may be labeled with a detectable label. The term “labeled”, with regard to the binding agent or antibody, is intended to encompass direct labeling of the binding agent or antibody by coupling (i.e., physically linking) a detectable substance to the binding agent or antibody, as well as indirect labeling of the binding agent or antibody by reactivity with another reagent that is directly labeled. An example of indirect labeling includes detection of a primary antibody using a fluorescently labeled secondary antibody. In one embodiment, the antibody is labeled, e.g., radio-labeled, chromophore-labeled, fluorophore-labeled, or enzyme-labeled. In another embodiment, the antibody is an antibody derivative (e.g., an antibody conjugated with a substrate or with the protein or ligand of a protein-ligand pair (e.g., biotin-streptavidin), or an antibody fragment (e.g., a single-chain antibody, an isolated antibody hypervariable domain).

Levels of a specific molecular marker (e.g., CA125, FRA, SA) may be determined by any means known in the art. In one embodiment, proteomic methods, e.g., mass spectrometry, are used. Mass spectrometry is an analytical technique that consists of ionizing chemical compounds to generate charged molecules (or fragments thereof) and measuring their mass-to-charge ratios. In a typical mass spectrometry procedure, a sample is obtained from a subject, loaded onto the mass spectrometry, and its components (e.g., CA125, FRA, SA) are ionized by different methods (e.g., by impacting them with an electron beam), resulting in the formation of charged particles (ions). The mass-to-charge ratio of the particles is then calculated from the motion of the ions as they transit through electromagnetic fields.

For example, matrix-associated laser desorption/ionization time-of-flight mass spectrometry (MALDI-TOF MS) or surface-enhanced laser desorption/ionization time-of-flight mass spectrometry (SELDI-TOF MS) which involves the application of a sample, such as urine or serum, to a protein-binding chip (Wright, G. L., Jr., et al. (2002) Expert Rev Mol Diagn 2:549; Li, J., et al. (2002) Clin Chem 48:1296; Laronga, C., et al. (2003) Dis Markers 19:229; Pctricoin, E. F., at al. (2002) 359:572; Adam, B. L., et al. (2002) Cancer Res 62:3609; Tolson, J., et al. (2004) Lab Invest 84:845; Xiao, Z., et al. (2001) Cancer Res 61:6029) can be used to determine the level of FRA.

Furthermore, in viva techniques for determination of the level of a molecular marker (e.g., CA125, FRA, SA) include introducing into a subject a labeled antibody directed against marker, which binds to and transforms the marker into a detectable molecule. The presence, level, or location of the detectable marker in a subject may be determined using standard imaging techniques.

As used herein, a “folate receptor-alpha-expressing ovarian cancer” includes any type of ovarian cancer characterized in that the cancer cells express or present on their surface folate receptor alpha. An ovarian cancer may have been, but is not required to have been, clinically diagnosed as expressing FRA to be encompassed by the term “folate receptor-alpha-expressing ovarian cancer” as used herein. The term also includes primary peritoneal or fallopian tube malignancies.

As used herein, a subject who is “afflicted with” or “having ovarian cancer” is one who is clinically diagnosed with ovarian cancer at any stage by a qualified clinician, or one who exhibits one or more signs or symptoms of such a cancer and is subsequently clinically diagnosed with such a cancer by a qualified clinician. A non-human subject that serves as an animal model of folate receptor-alpha-expressing ovarian cancer may also fall within the scope of a subject “afflicted with folate receptor-alpha-expressing ovarian cancer.”

The term “baseline level” with respect to a molecular marker refers to an initial determination of the amount or level of that marker in a subject or a biological sample obtained from a subject. For example, a baseline level of a biomarker may be the level of the marker determined upon or following diagnosis with ovarian cancer, upon or following surgical resection of the ovarian cancer, or upon or following initiation or completion of a first-line or other therapy for ovarian cancer.

The term “sample” as used herein refers to a collection of similar fluids, cells, or tissues isolated from a subject, as well as fluids, cells, or tissues present within a subject. Biological fluids are typically liquids at physiological temperatures and may include naturally occurring fluids present in, withdrawn from, expressed or otherwise extracted from a subject or biological source. Certain biological fluids derive from particular tissues, organs or localized regions and certain other biological fluids may be more globally or systemically situated in a subject or biological source. Examples of biological fluids include blood, serum and serosal fluids, plasma, lymph, urine, cerebrospinal fluid, saliva, ocular fluids, cystic fluid, tear drops, faces, sputum, mucosal secretions of the secretory tissues and organs, vaginal secretions, gynecological fluids, ascites fluids such as those associated with non-solid tumors, fluids of the pleural, pericardial, peritoneal, abdominal and other body cavities, fluids collected by bronchial lavage and the like. Biological fluids may also include liquid solutions contacted with a subject or biological source, for example, cell and organ culture medium including cell or organ conditioned medium, lavage fluids and the like.

In some embodiments, only a portion of the sample is subjected to an assay for determining the level of a molecular marker, or various portions of the sample are subjected to various assays for determining the level of a molecular marker. Also, in many embodiments, the sample may be pre-treated by physical or chemical means prior to the assay. For example, samples, may be subjected to centrifugation, dilution and/or treatment with a solubilizing substance (e.g., guanidine treatment) prior to assaying the samples for a molecular marker. Such techniques serve to enhance the accuracy, reliability and reproducibility of the assays.

The term “control sample,” as used herein, refers to any clinically relevant control sample, including, for example, a sample from a healthy subject not afflicted with ovarian cancer, a sample from a subject having a less severe or slower progressing ovarian cancer than the subject to be assessed, a sample from a subject having some other type of cancer or disease, and the like. A control sample may include a sample derived from one or more subjects. A control sample may also be a sample made at an earlier timepoint from the subject to be assessed. For example, the control sample could be a sample taken from the subject to be assessed before the onset of ovarian cancer, at an earlier stage of disease, or before the administration of treatment or of a portion of treatment. The control sample may also be a sample from an animal model, or from a tissue or cell lines derived from the animal model, of the ovarian cancer. The level of a molecular marker in a control sample that consists of a group of measurements may be determined based on any appropriate statistical measure, such as, for example, measures of central tendency including average, median, or modal values.

The term “control level” refers to an accepted or pre-determined level of a molecular marker which is used to compare with the level of the molecular marker in a sample derived from a subject. In one embodiment, the control level of a molecular marker is based on the level of the molecular marker in sample(s) from a subject(s) having slow disease progression. In another embodiment, the control level of a molecular marker is based on the level in a sample from a subject(s) having rapid disease progression. In another embodiment, the control level of a molecular marker is based on the level of the molecular marker in a sample(s) from an unaffected, i.e., non-diseased, subject(s), i.e., a subject who does not have ovarian cancer. In yet another embodiment, the control level of a molecular marker is based on the level of the molecular marker in a sample from a subject(s) prior to the administration of a therapy for ovarian cancer. In another embodiment, the control level of a molecular marker is based on the level of the molecular marker in a sample(s) from a subject(s) having ovarian cancer that is not contacted with a test compound. In another embodiment, the control level of a molecular marker is based on the level of the molecular marker in a sample(s) from a subject(s) not having ovarian cancer that is contacted with a test compound. In one embodiment, the control level of a molecular marker is based on the level of the molecular marker in a sample(s) from an animal model of ovarian cancer, a cell, or a cell line derived from the animal model of ovarian cancer.

In one embodiment, the control is a standardized control, such as, for example, a control which is predetermined using an average of the levels of a molecular marker from a population of subjects having no ovarian cancer. In still other embodiments of the invention, a control level of a molecular marker is based on the level of the molecular marker in a non-cancerous sample(s) derived from the subject having ovarian cancer. For example, when a laparotomy or other medical procedure reveals the presence of ovarian cancer in one portion of the ovaries, the control level of a molecular marker may be determined using the non-affected portion of the ovaries, and this control level may be compared with the level of the molecular marker in an affected portion of the ovaries.

As used herein, “a difference” between the level of a molecular marker in a sample from a subject (i.e., a test sample) and the level of the molecular marker in a control sample refers broadly to any clinically relevant and/or statistically significant difference in the level of the molecular marker in the two samples. For example, “an increase” in the level of a molecular marker may refer to a level in a test sample that is about two, and more preferably about three, about four, about five, about six, about seven, about eight, about nine, about ten or more times more than the level of the molecular marker in the control sample. An increase may also refer to a level in a test sample that is preferably at least about 1.5, and more preferably about two, about three, about four, about five or more standard deviations above the average level of the molecular marker in the control sample.

As used herein, the term “contacting the sample” with a specific binding agent, e.g., an antibody, includes exposing the sample, or any portion thereof with the agent or antibody, such that at least a portion of the sample comes into contact with the agent or antibody. The sample or portion thereof may be altered in some way, such as by subjecting it to physical or chemical treatments (e.g., dilution or guanidine treatment), prior to the act of contacting it with the agent or antibody.

The term “inhibit” or “inhibition of” means to reduce by a measurable amount, or to prevent entirely.

The term “deplete,” in the context of the effect of an anti-FRA therapeutic agent on folate receptor alpha-expressing cells, refers to a reduction in the number of, or elimination of, the folate receptor alpha-expressing cells.

The term “functional,” in the context of an antibody to be used in accordance with the methods described herein, indicates that the antibody is (1) capable of binding to antigen and/or (2) depletes or inhibits the proliferation of antigen-expressing cells.

The terms “treatment” or “treat” or “positive therapeutic response” refer to slowing, stopping, or reversing the progression of a folate receptor alpha-expressing ovarian cancer in a patient, as evidenced by a decrease or elimination of a clinical or diagnostic symptom of the disease, by administration of an anti-folate receptor alpha therapeutic agent to the subject after the onset of a clinical or diagnostic symptom of the folate receptor alpha-expressing ovarian cancer at any clinical stage. Treatment can include, for example, a decrease in the severity of a symptom, the number of symptoms, or frequency of relapse.

The phrase “responsive to treatment with an anti-FRA therapeutic agent” is intended to mean that the candidate subject (i.e., an individual with ovarian cancer), following administration of the anti-FRA therapeutic agent, would have a positive therapeutic response with respect to the ovarian cancer.

The term “pharmaceutically acceptable” refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability and includes properties and/or substances approved by a regulatory agency of the Federal or a state government or listed in the U.S. Pharmacopeia or other generally recognized pharmacopeia for use in animals, and more particularly in humans. The term “pharmaceutically compatible ingredient” refers to a pharmaceutically acceptable diluent, adjuvant, excipient, or vehicle with which an anti-folate receptor alpha antibody is administered. “Pharmaceutically acceptable carrier” refers to a medium that does not interfere with the effectiveness of the biological activity of the active ingredient(s) and is not toxic to the host to which it is administered.

The terms “effective amount” and “therapeutically effective amount” are used interchangeably herein and, in the context of the administration of a pharmaceutical agent, refer to the amount of the agent that is sufficient to inhibit the occurrence or ameliorate one or more clinical or diagnostic symptoms of a folate receptor alpha-expressing ovarian cancer in a patient. A therapeutically effective amount of an agent may vary according to factors such as the disease state, age, sex, and weight of the individual, and the ability of the antibody or antigen-binding fragment thereof to elicit a desired response in the individual. Such results may include, but are not limited to, the treatment of a folate-receptor alpha-expressing ovarian cancer, as determined by any means suitable in the art. An effective amount of an agent is administered according to the methods described herein in an “effective regimen.” The term “effective regimen” refers to a combination of amount of the agent and dosage frequency adequate to accomplish treatment of a folate receptor alpha-expressing ovarian cancer.

The terms “patient” and “subject” are used interchangeably to refer to humans and other non-human animals, including veterinary subjects, that receive diagnostic, prophylactic or therapeutic treatment. The term “non-human animal” includes all vertebrates, e.g., mammals and non-mammals, such as non-human primates, mice, rabbits, sheep, dog, cat, horse, cow, chickens, amphibians, and reptiles. In a preferred embodiment, the subject is a human.

Therapeutic agents are typically substantially pure from undesired contaminants. This means that an agent is typically at least about 50% w/w (weight/weight) pure as well as substantially free from interfering proteins and contaminants. Sometimes the agents are at least about 80% w/w and, more preferably at least 90 or about 95% w/w pure. However, using conventional protein purification techniques, homogeneous peptides of at least 99% purity w/w can be obtained.

Methods for Identifying a Subject Having Ovarian Cancer that Will be Responsive to Treatment with an Anti-FRA Therapeutic Agent

Provided herein are methods for identifying a subject having a folate receptor alpha (FRA)-expressing ovarian cancer that will be responsive to treatment with an anti-FRA therapeutic agent. In some embodiments of the methods for identifying a subject having ovarian cancer that will be responsive to treatment with an anti-FRA therapeutic agent described herein, the ovarian cancer that will be responsive to treatment with an anti-folate receptor alpha (FRA) therapeutic agent is epithelial ovarian cancer. In some embodiments, the ovarian cancer is either platinum-sensitive or platinum-resistant. The subject may have received a platinum-based or platinum- and taxane-based first-line therapy.

The methods for identifying a subject having a folate receptor alpha (FRA)-expressing ovarian cancer that will be responsive to treatment with an anti-FRA therapeutic agent as described herein involve determining a baseline level of cancer antigen 125 (CA125) expression of the subject. To date, CA125 is the most commonly measured tumor marker for epithelial ovarian tumors, which account for 85-90% of ovarian cancers. CA125, however, is only elevated in 47% of women with early-stage ovarian cancer, while CA125 levels are elevated in 80-90% of advanced-stage ovarian cancers [American College of Obstetricians and Gynecologists. PROLOG Gynecology and Surgery (6th Edition). American College of Obstetricians and Gynecologists, Washington, D.C., USA (2009)]. As is understood by those skilled in the art, the upper limit of normal (ULN) for CA125 varies depending upon the assay employed. For example, the upper limit of normal for CA125 in the Immulite® assay for CA125 exemplified herein is currently established to be about 21 units per milliliter (U/ml). In other such CA125 assays, however, exemplified by the Abbott Architect, Beckman Access and the like, the upper limit of normal for CA125 is established to be about 35 U/ml. In the methods for identifying a subject having a folate receptor alpha (FRA)-expressing ovarian cancer that will be responsive to treatment with an anti-FRA therapeutic agent provided herein, a baseline CA125 level that is less than about eight times the upper limit of normal (ULN) for CA125 is indicative of a subject who would benefit from treatment with an anti-FRA therapeutic agent. In some embodiments, a baseline CA125 level that is less than about seven times the ULN for CA125, more preferably less than about six times the ULN for CA125, more preferably less than about five times the ULN for CA125, more preferably less than about four times the ULN for CA125, even more preferably less than about three times the ULN for CA125, and even more preferably less than about two times the ULN for CA125 is indicative of a subject who would benefit from treatment with an anti-FRA therapeutic agent. In some embodiments, a baseline CA125 level that is less than about the ULN for CA125 is indicative of a subject who would benefit from treatment with an anti-FRA therapeutic agent. In some embodiments, a baseline CA125 level that is less than about 164 units/ml, preferably less than about 150 units/ml, more preferably less than about 140 units/ml, more preferably less than about 130 units/ml, more preferably less than about 120 units/ml, more preferably less than about 110 units/ml, more preferably less than about 100 units/ml, even more preferably less than about 90 units/ml, more preferably less than about 80 units/ml, more preferably less than about 70 units/ml, more preferably less than about 63 units/ml, in some embodiments less than about 42 units/ml, in some embodiments less than about 35 units/ml, and in some embodiments, less than about 21 units/ml, is indicative of a subject who would benefit from treatment with an anti-FRA therapeutic agent.

In the methods for identifying a subject having ovarian cancer that will be responsive to treatment with an anti-FRA therapeutic agent described herein, CA125 expression level may be determined by any means known in the art. For example, the level of CA125 expression may be determined using an antibody to detect protein expression, nucleic acid hybridization, quantitative RT-PCR, western blot analysis, radioimmunoassay, immunofluorimetry, immunoprecipitation, equilibrium dialysis, immunodiffusion, electrochemiluminescence (ECL) immunoassay, immunohistochemistry, fluorescence-activated cell sorting (FACS), or ELISA assay. The step of determining expression level of CA125 may be performed ex vivo or in vivo.

For ex vivo assessments, the biological sample used in determining the baseline level of CA125 may be may be derived from whole blood, serum, plasma, pleural effusions, ascites, tissues (e.g., surgically resected tumor tissue, biopsies, including fine needle aspiration), histological preparations, and the like. The sample on which the assay is performed can be fixed or frozen to permit histological sectioning. Preferably, the excised tissue samples are fixed in aldehyde fixatives such as formaldehyde, paraformaldehyde, glutaraldehyde; or heavy metal fixatives such as mercuric chloride. More preferably, the excised tissue samples are fixed in formalin and embedded in paraffin wax prior to incubation with the antibody. Optionally, FFPE specimens can be treated with citrate, EDTA, enzymatic digestion or heat to increase accessibility of epitopes. Alternatively, a protein fraction can be isolated from cells from known or suspected ovarian cancer and analyzed by ELISA, Western blotting, immunoprecipitation or the like. In another variation, cells can be analyzed for expression of folate receptor alpha by FACS analysis. In a further variation, mRNA can be extracted from cells from known or suspected ovarian cancer. The mRNA or a nucleic acid derived therefrom, such as a cDNA can then be analyzed by hybridization to a nucleic probe binding to DNA encoding folate receptor alpha.

For example, the step of determining expression level of CA125 may involve determining the level of CA125 expression in a biological sample of the ovarian cancer tissue obtained from the subject. CA125 expression levels may be determined by an immunoassay in which a sample containing cells known or suspected to be from a cancer (e.g., ovarian cancer) is contacted with an anti-CA125 antibody or antigen-binding fragment After contact, the presence or absence of a binding event of the antibody or antigen-binding fragment to the cells in the specimen is determined. The binding is related to the presence or absence of the antigen expressed on cancerous cells in this specimen. Generally, the sample is contacted with a labeled specific binding partner of the anti-CA125 antibody or antigen-binding fragment capable of producing a detectable signal. Alternatively, the anti-CA125 antibody or fragment itself can be labeled. Examples of types of labels include enzyme labels, radioisotopic labels, nonradioactive labels, fluorescent labels, toxin labels and chemoluminescent labels. Many such labels are readily known to those skilled in the art. For example, suitable labels include, but should not be considered limited to, radiolabels, fluorescent labels (such as DyLight®649), epitope tags, biotin, chromophore labels, ECL labels, or enzymes. More specifically, the described labels include ruthenium, ¹¹¹In-DOTA, ¹¹¹In-diethylenetriaminepentaacetic acid (DTPA), horseradish peroxidase, alkaline phosphatase and beta-galactosidase, poly-histidine (HIS tag), acridine dyes, cyanine dyes, fluorone dyes, oxazin dyes, phenanthridine dyes, rhodamine dyes, Alexafluor® dyes, and the like. Detection of a signal from the label indicates the presence of the antibody or fragment specifically bound to folate receptor alpha in the sample.

In another variation, CA125 expression level in known or suspected ovarian cancer can be detected in vivo by administering a labeled anti-CA125 antibody or antigen-binding fragment thereof to a patient and detecting the antibody or fragment by in vivo imaging.

The level of CA125 in an ovarian tissue sample can (but need not) be determined with respect to one or more standards. The standards can be historically or contemporaneously determined. The standard can be, for example, an ovarian tissue sample known not to be cancerous from a different subject, a tissue from either the patient or other subject known not to express CA125, or an ovarian cell line. The standard can also be the patient sample under analysis contacted with an irrelevant antibody (e.g., an antibody raised to a bacterial antigen).

The presence of detectable signal from binding of an anti-CA125 antibody or fragment to CA125 relative to a standard (if used) indicates the presence of CA125 in the tissue sample, and the level of detectable binding provides an indication of the level of expression of CA125. In assays performed on tissue sections, the level of expression can be expressed as a percentage of the surface area of the sample showing detectable expression of CA125. Alternatively, or additionally, the level (intensity) of expression can be used as a measure of the total expression in the sample or of the cells expressing CA125 in the sample.

The baseline level of CA125 may be either a measurement of the CA125 level in the subject at a single timepoint or may involve measurement of CA125 levels in the subject at two, three, four, five, or more points in time (e.g., serial CA125 determinations). Determination of the baseline level of CA125 in the subject may be performed upon diagnosis, upon surgical resection, upon initiation of first-line therapy, upon completion of first-line therapy, upon initiation of second-line therapy, upon completion of second-line therapy, and/or upon symptomatic progression, serologic progression, and/or radiologic progression of the cancer.

Some embodiments of the methods for identifying a subject having ovarian cancer that will be responsive to treatment with an anti-FRA therapeutic agent by determining a baseline level of cancer antigen 125 (CA125) expression of the subject further involve determining the level of FRA in a sample derived from the subject; wherein an increase in the level of FRA in the sample derived from said subject as compared to the level of FRA in the control sample is indicative that the subject would benefit from treatment with an anti-FRA therapeutic agent.

In some embodiments, the level of FRA in a sample is assessed by contacting the sample with an antibody that binds FRA. Antibodies that bind FRA are known in the art and include (i) the murine monoclonal LK26 antibody, the heavy and light chains of which are presented herein as SEQ ID NOs: 11 and 12, respectively:

SEQ ID NO: 11
Gln Val Xaa Leu Gln Xaa Ser Gly Gly Asp Leu Val
Lys Pro Gly Gly Ser Leu Lys Leu Ser Cys Ala Ala
Ser Gly Phe Thr Phe Ser Gly Tyr Gly Leu Ser Trp
Val Arg Gln Thr Pro Asp Lys Arg Leu Glu Trp Val
Ala Met Ile Ser Ser Gly Gly Ser Tyr Thr Tyr Tyr
Ala Asp Ser Val Lys Gly Arq Phe Ala Ile Ser Arg
Asp Asn Ala Lys Asn Ser Leu Phe Leu Gln Met Ser
Ser Leu Lys Ser Asp Asp Thr Ala Ile Tyr Ile Cys
Ala Arg His Gly Asp Asp Pro Ala Trp Phe Ala Tyr
Trp Gly Gln Gly Thr Leu Val Thr Val Ser Ala
(wherein Xaa refers to any amino acid)
SEQ ID NO: 12
Asp Ile Glu Leu Thr Gln Ser Pro Ala Leu Met Ala
Ala Ser Pro Gly Glu Lys Val Thr Ile Thr Cys Ser
Val Ser Ser Ser Ile Ser Ser Asn Asn Leu His Trp
Tyr Gln Gln Lys Ser Glu Thr Ser Pro Lys Pro Trp
Ile Tyr Gly Thr Ser Asn Leu Ala Ser Gly Val Pro
Leu Arg Phe Arg Gly Phe Gly Ser Gly Thr Ser Tyr
Ser Leu Thr Ile Ser Ser Met Glu Ala Glu Asp Ala
Ala Thr Tyr Tyr Cys Gln Gln Trp Ser Ser Tyr Pro
Tyr Met Tyr Thr Phe Gly Gly Gly Thr Lys Leu Glu
Ile Lys

as described in European Patent Application No. 86104170.5 (Rettig) (the entire contents of which are incorporated herein by reference); (ii) the MORAB-003 antibody, as described in U.S. Publ. No. 20090274697 and U.S. Pat. No. 8,124,083, the entire contents of each of which are incorporated herein by reference. The monoclonal antibodies MOV18 and MOv19 also bind different epitopes on the FRα molecule (previously known as gp38/FBP). Miotti, S. et al. Int J Cancer, 38: 297-303 (1987). For example, the MOV18 antibody binds the epitope set forth herein as SEQ ID NO: 13 (TELLNVXMNAK*XKEKPXPX*KLXXQX) (note that at position 12, a tryptophan or histidine residue is possible, and at position 21, an aspartic acid or glutamic acid residue is possible), as taught in Coney et al. Cancer Res, 51: 6125-6132 (1991).

In some embodiments, the FRA is not bound to a cell in the sample. Methods for determining the level of FRA in a sample derived from the subject are disclosed, for example, in U.S. Publ. No. 20130017195, incorporated herein by reference. Methods for determining the level of FRA which is not bound to a cell in a sample derived from the subject are disclosed, for example, in U.S. Publ. No. 20120207771, incorporated herein by reference. The sample employed in the determination of the level of FRA may be tissue (e.g., tumor biopsy), urine, serum, plasma or ascites, for example. In preferred embodiments, the sample is tissue or serum. In various aspects, the level of FRA is determined by contacting the sample with an antibody that binds FRA. For example, the antibody is selected from the group consisting of:

(a) an antibody that binds the same epitope as the MORAb-003 antibody;

(b) an antibody comprising SEQ ID NO:1 (GFTFSGYGLS) as CDRH1, SEQ ID NO:2 (MISSGGSYTYYADSVKG) as CDRH2, SEQ ID NO:3 (HGDDPAWFAY) as CDRH3, SEQ ID NO:4 (SVSSSISSNNLH) as CDRL1, SEQ ID NO:5 (GTSNLAS) as CDRL2 and SEQ ID NO:6 (QQWSSYPYMYT) as CDRL3;

(c) the MOV18 antibody;

(d) an antibody that binds the same epitope as the MOV18 antibody;

(e) the 548908 antibody;

(f) an antibody that binds the same epitope as the 548908 antibody;

(g) the 6D398 antibody;

(h) an antibody that binds the same epitope as the 6D398 antibody;

(i) an antibody that binds the same epitope as the 26B3 antibody;

(j) an antibody comprising SEQ ID NO: 14 (GYFMN) as CDRH1, SEQ ID NO: 15 (RIFPYNGDTFYNQKFKG) as CDRH2, SEQ ID NO: 16 (GTHYFDY) as CDRH3, SEQ ID NO: 17 (RTSENIFSYLA) as CDRL1, SEQ ID NO: 18 (NAKTLAE) as CDRL2 and SEQ ID NO: 19 (QHHYAFPWT) as CDRL3;

(k) the 26B3 antibody;

(l) an antibody that binds the same epitope as the 19D4 antibody;

(m) an antibody comprising SEQ ID NO: 20 (HPYMH) as CDRH1, SEQ ID NO: 21 (RIDPANGNTKYDPKFQG) as CDRH2, SEQ ID NO: 22 (EEVADYTMDY) as CDRH3, SEQ ID NO: 23 (RASESVDTYGNNFIH) as CDRL1, SEQ ID NO: 24 (LASNLES) as CDRL2 and SEQ ID NO: 25 (QQNNGDPWT) as CDRL3;

(n) the 19D4 antibody;

(o) an antibody that binds the same epitope as the 9F3 antibody;

(p) an antibody comprising SEQ ID NO: 26 (SGYYWN) as CDRH1, SEQ ID NO: 27 (YIKSDGSNNYNPSLKN) as CDRH2, SEQ ID NO: 28 (EWKAMDY) as CDRH3, SEQ ID NO: 29 (RASSTVSYSYLH) as CDRL1, SEQ ID NO: 30 (GTSNLAS) as CDRL2 and SEQ ID NO: 31 (QQYSGYPLT) as CDRL3;

(q) the 9F3 antibody;

(r) an antibody that binds the same epitope as the 24F12 antibody;

(s) an antibody comprising SEQ ID NO: 32 (SYAMS) as CDRH1, SEQ ID NO: 33 (EIGSGGSYTYYPDTVTG) as CDRH2, SEQ ID NO: 34 (ETTAGYFDY) as CDRH3, SEQ ID NO: 35 (SASQGINNFLN) as CDRL1, SEQ ID NO: 36 (YTSSLHS) as CDRL2 and SEQ ID NO: 37 (QHFSKLPWT) as CDRL3;

(t) the 24F12 antibody;

(u) an antibody that comprises a variable region light chain selected from the group consisting of LK26HuVK as set forth in SEQ ID NO: 38:

Asp Ile Gln Leu Thr Gln Ser Pro Ser Ser Leu Ser
Ala Ser Val Gly Asp Arg Val Thr Ile Thr Cys Ser
Val Ser Ser Ser Ile Ser Ser Asn Asn Leu His Trp
Tyr Gln Gln Lys Pro Gly Lys Ala Pro Lys Leu Leu
Ile Tyr Gly Thr Ser Asn Leu Ala Ser Gly Val Pro
Ser Arg Phe Ser Gly Ser Gly Ser Gly Thr Asp Phe
Thr Phe Thr Ile Ser Ser Leu Gln Pro Glu Asp Ile
Ala Thr Tyr Tyr Cys Gln Gln Trp Ser Ser Tyr Pro
Tyr Met Tyr Thr Phe Gly Gln Gly Thr Lys Val Glu
Ile Lys,

LK26HuVKY as set forth in SEQ ID NO: 39:

Asp Ile Gln Leu Thr Gln Ser Pro Ser Ser Leu Ser
Ala Ser Val Gly Asp Arg Val Thr Ile Thr Cys Ser
Val Ser Ser Ser Ile Ser Ser Asn Asn Leu His Trp
Tyr Gln Gln Lys Pro Gly Lys Ala Pro Lys Leu Leu
Ile Tyr Gly Thr Ser Asn Leu Ala Ser Gly Val Pro
Ser Arg Phe Ser Gly Ser Gly Ser Gly Thr Asp Tyr
Thr Phe Thr Ile Ser Ser Leu Gln Pro Glu Asp Ile
Ala Thr Tyr Tyr Cys Gln Gln Trp Ser Ser Tyr Pro
Tyr Met Tyr Thr Phe Gly Gln Gly Thr Lys Val Glu
Ile Lys,

LK26HuVKPW as set forth in SEQ ID NO: 40:

Asp Ile Gln Leu Thr Gln Ser Pro Ser Ser Leu Ser
Ala Ser Val Gly Asp Arg Val Thr Ile Thr Cys Ser
Val Ser Ser Ser Ile Ser Ser Asn Asn Leu His Trp
Tyr Gln Gln Lys Pro Gly Lys Ala Pro Lys Pro Trp
Ile Tyr Gly Thr Ser Asn Leu Ala Ser Gly Val Pro
Ser Arg Phe Ser Gly Ser Gly Ser Gly Thr Asp Phe
Thr Phe Thr Ile Ser Ser Leu Gln Pro Glu Asp Ile
Ala Thr Tyr Tyr Cys Gln Gln Trp Ser Ser Tyr Pro
Tyr Met Tyr Thr Phe Gly Gln Gly Thr Lys Val Glu
Ile Lys,

and
LK26HuVKPW,Y as set forth in SEQ ID NO: 41:

Asp Ile Gln Leu Thr Gln Ser Pro Ser Ser Leu Ser
Ala Ser Val Gly Asp Arg Val Thr Ile Thr Cys Ser
Val Ser Ser Ser Ile Ser Ser Asn Asn Leu His Trp
Tyr Gln Gln Lys Pro Gly Lys Ala Pro Lys Pro Trp
Ile Tyr Gly Thr Ser Asn Leu Ala Ser Gly Val Pro
Ser Arg Phe Ser Gly Ser Gly Ser Gly Thr Asp Tyr
Thr Phe Thr Ile Ser Ser Leu Gln Pro Glu Asp Ile
Ala Thr Tyr Tyr Cys Gln Gln Trp Ser Ser Tyr Pro
Tyr Met Tyr Thr Phe Gly Gln Gly Thr Lys Val Glu
Ile Lys;

(v) an antibody that comprises a variable region heavy chain selected from the group consisting of LK26HuVH as set forth in SEQ ID NO: 42:

Gln Val Gln Leu Gln Glu Ser Gly Pro Gly Leu Val
Arg Pro Ser Gln Thr Leu Ser Leu Thr Cys Thr Ala
Ser Gly Phe Thr Phe Ser Gly Tyr Gly Leu Ser Trp
Val Arg Gln Pro Pro Gly Arg Gly Leu Glu Trp Val
Ala Met Ile Ser Ser Gly Gly Ser Tyr Thr Tyr Tyr
Ala Asp Ser Val Lys Gly Arg Val Thr Met Leu Arg
Asp Thr Ser Lys Asn Gln Phe Ser Leu Arg Leu Ser
Ser Val Thr Ala Ala Asp Thr Ala Val Tyr Tyr Cys
Ala Arg His Gly Asp Asp Pro Ala Trp Phe Ala Tyr
Trp Gly Gln Gly Ser Leu Val Thr Val Ser Ser,

LK26HuVH FAIS,N as set forth in SEQ ID NO: 43:

Gln Val Gln Leu Gln Glu Ser Gly Pro Gly Leu Val
Arg Pro Ser Gln Thr Leu Ser Leu Thr Cys Thr Ala
Ser Gly Phe Thr Phe Ser Gly Tyr Gly Leu Ser Trp
Val Arg Gln Pro Pro Gly Arg Gly Leu Glu Trp Val
Ala Met Ile Ser Ser Gly Gly Ser Tyr Thr Tyr Tyr
Ala Asp Ser Val Lys Gly Arg Phe Ala Ile Ser Arg
Asp Asn Ser Lys Asn Gln Phe Ser Leu Arg Leu Ser
Ser Val Thr Ala Ala Asp Thr Ala Val Tyr Tyr Cys
Ala Arg His Gly Asp Asp Pro Ala Trp Phe Ala Tyr
Trp Gly Gln Gly Ser Leu Val Thr Val Ser Ser,

LK26HuVH SLF as set forth in SEQ ID NO: 44:

Gln Val Gln Leu Gln Glu Ser Gly Pro Gly Leu Val
Arg Pro Ser Gln Thr Leu Ser Leu Thr Cys Thr Ala
Ser Gly Phe Thr Phe Ser Gly Tyr Gly Leu Ser Trp
Val Arg Gln Pro Pro Gly Arg Gly Leu Glu Trp Val
Ala Met Ile Ser Ser Gly Gly Ser Tyr Thr Tyr Tyr
Ala Asp Ser Val Lys Gly Arg Val Thr Met Leu Arg
Asp Thr Ser Lys Asn Ser Leu Phe Leu Arg Leu Ser
Ser Val Thr Ala Ala Asp Thr Ala Val Tyr Tyr Cys
Ala Arg His Gly Asp Asp Pro Ala Trp Phe Ala Tyr
Trp Gly Gln Gly Thr Thr Val Thr Val Ser Ser,

LK26HuVH I,I as set forth in SEQ ID NO: 45:

Gln Val Gln Leu Gln Glu Ser Gly Pro Gly Leu Val
Arg Pro Ser Gln Thr Leu Ser Leu Thr Cys Thr Ala
Ser Gly Phe Thr Phe Ser Gly Tyr Gly Leu Ser Trp
Val Arg Gln Pro Pro Gly Arg Gly Leu Glu Trp Val
Ala Met Ile Ser Ser Gly Gly Ser Tyr Thr Tyr Tyr
Ala Asp Ser Val Lys Gly Arg Val Thr Met Leu Arg
Asp Thr Ser Lys Asn Gln Phe Ser Leu Arg Leu Ser
Ser Val Thr Ala Ala Asp Thr Ala Ile Tyr Ile Cys
Ala Arg His Gly Asp Asp Pro Ala Trp Phe Ala Tyr
Trp Gly Gln Gly Ser Leu Val Thr Val Ser Ser,

and LK26KOLHuVH as set forth in SEQ ID NO: 46:

Glu Val Gln Leu Val Glu Ser Gly Gly Gly Val Val
Gln Pro Gly Arg Ser Leu Arg Leu Ser Cys Ser Ala
Ser Gly Phe Thr Phe Ser Gly Tyr Gly Leu Ser Trp
Val Arg Gln Ala Pro Gly Lys Gly Leu Glu Trp Val
Ala Met Ile Ser Ser Gly Gly Ser Tyr Thr Tyr Tyr
Ala Asp Ser Val Lys Gly Arg Phe Ala Ile Ser Arg
Asp Asn Ala Lys Asn Thr Leu Phe Leu Gln Met Asp
Ser Leu Arg Pro Glu Asp Thr Gly Val Tyr Phe Cys
Ala Arg His Gly Asp Asp Pro Ala Trp Phe Ala Tyr
Trp Gly Gln Gly Thr Pro Val Thr Val Ser Ser;

(w) an antibody that comprises the heavy chain variable region LK26KOLHuVH (SEQ ID NO: 46) and the light chain variable region LK26HuVKPW,Y (SEQ ID NO: 41);

(x) an antibody that comprises the heavy chain variable region LK26HuVH SLF (SEQ ID NO: 44) and the light chain variable region LK26HuVKPW,Y (SEQ ID NO: 41); and

(y) an antibody that comprises the heavy chain variable region LK26HuVH FAIS,N (SEQ ID NO: 43) and the light chain variable region LK26HuVKPW,Y (SEQ ID NO: 41).

In a particular embodiment, the antibody binds the same epitope as the MORAb-003 antibody. In another embodiment, the antibody includes SEQ ID NO:1 (GFTFSGYGLS) as CDRH1, SEQ ID NO:2 (MISSGGSYTYYADSVKG) as CDRH2, SEQ ID NO:3 (HGDDPAWFAY) as CDRH3, SEQ ID NO:4 (SVSSSISSNNLH) as CDRL1, SEQ ID NO:5 (GTSNLAS) as CDRL2 and SEQ ID NO:6 (QQWSSYPYMYT) as CDRL3. In another embodiment, the antibody is the MOV18 antibody. In yet another embodiment, the antibody binds the same epitope as the MOV18 antibody. In a further embodiment, the antibody comprises a variable region light chain selected from the group consisting of LK26HuVK (SEQ ID NO: 38); LK26HuVKY (SEQ ID NO: 39); LK26HuVKPW (SEQ ID NO: 40); and LK26HuVKPW,Y (SEQ ID NO: 41). Alternatively or in combination, the antibody includes a variable region heavy chain selected from the group consisting of LK26HuVH (SEQ ID NO: 42); LK26HuVH FAIS,N (SEQ ID NO: 43); LK26HuVH SLF (SEQ ID NO: 44); LK26HuVH I,I (SEQ ID NO: 45); and LK26KOLHuVH (SEQ ID NO: 46). In certain embodiments, the antibody includes (i) the heavy chain variable region LK26KOLHuVH (SEQ ID NO: 46) and the light chain variable region LK26HuVKPW,Y (SEQ ID NO: 41); the heavy chain variable region LK26HuVH SLF (SEQ ID NO: 44) and the light chain variable region LK26HuVKPW,Y (SEQ ID NO: 41); or the heavy chain variable region LK26HuVH FAIS,N (SEQ ID NO: 43) and the light chain variable region LK26HuVKPW,Y (SEQ ID NO: 41).

In a particular embodiment, the level of FRA in the sample derived from said subject is assessed by contacting the sample with a pair of antibodies selected from the group consisting of (a) MOV18 antibody immobilized to a solid support and labeled MORAB-003 antibody; (b) 9F3 antibody immobilized to a solid support and labeled 24F12 antibody; (c) 26B3 antibody immobilized to a solid support and labeled 19D4 antibody; and (d) 9F3 antibody immobilized to a solid support and labeled 26B3 antibody.

In certain embodiments, the antibody is selected from the group consisting of a murine antibody, a human antibody, a humanized antibody, a bispecific antibody, a chimeric antibody, a Fab, Fab′2, ScFv, SMIP, affibody, avimer, versabody, nanobody, and a domain antibody. Alternatively, or in combination, the antibody is labeled, for example, with a label selected from the group consisting of a radio-label, a biotin-label, a chromophore-label, a fluorophore-label, or an enzyme-label.

In certain embodiments, the level of FRA is determined by western blot analysis, radioimmunoassay, immunofluorimetry, immunoprecipitation, equilibrium dialysis, immunodiffusion, solution phase assay, electrochemiluminescence immunoassay (ECLIA) or ELISA assay.

In various embodiments of the foregoing aspects of the invention, the control sample is a standardized control level of FRA in a healthy subject.

In certain embodiments, the sample is treated with guanidine prior to determining the level of FRA in the sample. Alternatively or in combination, the sample is diluted prior to determining the level of FRA in the sample. Alternatively, or in combination, the sample is centrifuged, vortexed, or both, prior to determining the level of FRA in the sample.

In a further aspect, the level of folate receptor alpha (FRA) in a sample derived from the subject is assessed by a two-antibody sandwich assay. In some embodiments of the sandwich assay, the sample is contacted with (a) MOV18 antibody immobilized to a solid support and labeled MORAB-003 antibody, (b) 9F3 antibody immobilized to a solid support and labeled 24F12 antibody, (c) 26B3 antibody immobilized to a solid support and labeled 19D4 antibody, and (d) 9F3 antibody immobilized to a solid support and labeled 26B3 antibody. For example, the sample may be urine, serum, plasma or ascites.

In some embodiments of the methods for identifying a subject having ovarian cancer that will be responsive to treatment with an anti-FRA therapeutic agent, the anti-FRA therapeutic agent is an antibody that specifically binds to folate receptor alpha, preferably to FRA expressed on ovarian cancer cells; antigen-binding fragments of such an antibody; derivatives; and variants thereof. An exemplary antibody that specifically binds to folate receptor alpha may be an antibody selected from the group consisting of:
(a) an antibody comprising SEQ ID NO: 1 (GFTFSGYGLS) as CDRH1, SEQ ID NO:2 (MISSGGSYTYYADSVKG) as CDRH2, SEQ ID NO:3 (HGDDPAWFAY) as CDRH3, SEQ ID NO:4 (SVSSSISSNNLH) as CDRL1, SEQ ID NO:5 (GTSNLAS) as CDRL2 and SEQ ID NO:6 (QQWSSYPYMYT) as CDRL3; or
(b) an antibody that binds the same epitope on farletuzumab
   In some embodiments, the antibody that specifically binds to folate receptor alpha comprises a mature light chain variable region comprising the amino acid sequence of SEQ ID NO:7:

| | |
|---|---|
| 1 | DIQLTQSPSS LSASVGDRVT ITCSVSSSIS SNNLHWYQQK PGKAPKPWIY |
| 51 | GTSNLASGVP SRFSGSGSGT DYTFTISSLQ PEDIATYYCQ QWSSYPYMYT |
| 101 | FGQGTKVEIK RTVAAPSVFI FPPSDEQLKS GTASVVCLLN NFYPREAKVQ |
| 151 | WKVDNALQ3G NSQESVTEQD SKDSTYSLSS TLTLSKADYE KHKVYACEVT |
| 201 | HQGLSSPVTK SFNRGEC |

### (CDRs Underlined).

In some embodiments, the antibody that specifically binds to folate receptor alpha comprises a mature heavy chain variable region comprising the amino acid SEQ ID NO: 8:

| | |
|---|---|
| 1 | EVQLVESGGG VVQPGRSLRL SCSASGFTFS GYGLSWVRQA PGKGLEWVAM |
| 51 | ISSGGSYTYY ADSVKGRFAI SRDNAKNTLF LQMDSLRPED TGVYFCARHG |
| 101 | DDPAWFAYWG QGTPVTVSSA STKGPSVFPL APSSKSTSGG TAALGCLVKD |
| 151 | YFPEPVTVSW NSGALTSGVH TFPAVLQSSG LYSLSSVVTV PSSSLGTQTY |
| 201 | ICNVNHKPSN TKVDKKVEPK SCDKTHTCPP CPAPELLGGP SVFLFPPKPK |
| 251 | DTLMISRTPE VTCVVVDVSH EDPEVKFNWY VDGVEVHNAK TKPREEQYNS |
| 301 | TYRVVSVLTV LHQDWLNGKE YKCKVSNKAL PAPIEKTISK AKGQPREPQV |
| 351 | YTLPPSRDEL TKNQVSLTCL VKGFYPSDIA VEWESNGQPE NNYKTTPPVL |
| 401 | DSDGSFFLYS KLTVDKSRWQ QGNVFSCSVM HEALHNHYTQ KSLSLSPGK |

### (CDRs Underlined).

In some embodiments, the antibody that specifically binds to folate receptor alpha comprises a mature light chain variable region comprising the amino acid sequence of SEQ ID NO: 7 and a mature heavy chain variable region comprising the amino acid sequence of SEQ ID NO: 8. An example of such an antibody is MORAb-003 (USAN: farletuzumab). Farletuzumab is a humanized monoclonal antibody directed against folate receptor a (FRA). It has been shown to mediate tumor cytotoxicity via antibody dependent cell cytotoxicity (ADCC) and complement dependent cytotoxicity (CDC) of a FRA-expressing human ovarian cancer cell line in vitro and to reduce tumor growth in FRA-expressing human ovarian cancer cells in vivo in a xenograft model (Ebel et al. (2007) Cancer Immun 7: 6). Chinese hamster ovary (CHO) cells producing MORAb-003 have been deposited with the ATCC (10801 University Boulevard, Manassas, Va. 20110) on Apr. 24, 2006 and assigned accession no. PTA-7552.

Other useful antibodies that specifically bind to folate receptor alpha comprise mature light and heavy chain variable regions having at least 90% and preferably at least 95% or 99% sequence identity to SEQ ID NO: 7 and SEQ ID NO: 8, respectively. Other useful anti-folate receptor alpha antibodies or derivatives thereof can competitively inhibit binding of farletuzumab to folate receptor alpha, as determined, for example, by immunoassay. Competitive inhibition means that an antibody when present in at least a two-fold and preferably five-fold excess inhibits binding of farletuzumab to folate receptor alpha by at least 50%, more typically at least 60%, yet more typically at least 70%, and most typically at least 75%, at least 80%, at least 85%, at least 91%, or at least 95%.

The anti-FRA therapeutic agent may also be a derivative of an anti-folate receptor alpha antibody. Typical modifications include, e.g., glycosylation, deglycosylation, acetylation, pegylation, phosphorylation, amidation, derivatization by known protecting/blocking groups, proteolytic cleavage, linkage to a cellular ligand or other protein, and the like. Additionally, the derivative may contain one or more non-classical amino acids.

In some embodiments of the methods for identifying a subject having ovarian cancer that will be responsive to treatment with an anti-FRA therapeutic agent described herein, the subject may have received surgical resection of the ovarian cancer, first-line platinum-based therapy, first-line taxane-based therapy, and/or first-line platinum and taxane-based therapy for treatment of the ovarian cancer prior to determining the baseline level of CA125. In some embodiments of the methods in which the subject received surgical resection of the ovarian cancer, first-line platinum-based therapy, first-line taxane-based therapy, and/or first-line platinum and taxane-based therapy for treatment of the ovarian cancer prior to determining the baseline level of CA125, the subject may have exhibited symptomatic progression, serologic progression, and/or radiologic progression of the ovarian cancer prior to the step of determining the baseline level of CA125.

In additional embodiments of the methods for identifying a subject having ovarian cancer that will be responsive to treatment with an anti-FRA therapeutic agent and methods of treatment described herein, a baseline serum albumin (SA) concentration of the subject is determined. Methods for determining serum albumin (SA) concentration are known in the art. A baseline SA concentration of at least about 2.0 g/dL, preferably at least about 3.0 g/dL, and even more preferably at least about 3.2 g/dL is further indicative of a positive therapeutic response to the anti-FRA therapeutic agent. The baseline level of SA may be either a measurement of the SA level in the subject at a single timepoint or may involve measurement of SA levels in the subject at at least two points in time. Determination of the baseline level of SA in the subject may be performed upon diagnosis, upon surgical resection, upon initiation of first-line therapy, upon completion of first-line therapy, upon symptomatic progression, serologic progression, and/or radiologic progression of the cancer, upon initiation of second-line therapy, and/or upon completion of second-line therapy.

Methods of Treatment

Also provided herein are methods of treating a subject with folate receptor alpha (FRA)-expressing ovarian cancer. In some embodiments of the methods for identifying a subject having ovarian cancer that will be responsive to treatment with an anti-FRA therapeutic agent described herein, the ovarian cancer that will be responsive to treatment with an anti-folate receptor alpha (FRA) therapeutic agent is epithelial ovarian cancer. In some embodiments, the ovarian cancer is either platinum-sensitive or platinum-resistant.

In accordance with the methods of treating a subject with folate receptor alpha (FRA)-expressing ovarian cancer described herein, the baseline level of CA125 in a biological sample obtained from the subject is determined. In some embodiments, when the baseline CA125 level is determined to be less than about eight times the ULN for CA125, preferably about seven times the ULN for CA125, more preferably less than about six times the ULN for CA125, more preferably less than about five times the ULN for CA125, more preferably less than about four times the ULN for CA125, more preferably less than about three times the ULN for CA125, and more preferably less than about two times the ULN for CA125 is indicative of a subject who would benefit from treatment with an anti-FRA therapeutic agent. In some embodiments, a baseline CA125 level that is less than about the ULN for CA125 is indicative of a subject who would benefit from treatment with an anti-FRA therapeutic agent. In some embodiments, when the CA125 level is determined to be less than about 164 units/ml, preferably less than about 150 units/ml, more preferably less than about 140 units/ml, more preferably less than about 130 units/ml, more preferably less than about 120 units/ml, more preferably less than about 110 units/ml, more preferably less than about 100 units/ml, even more preferably less than about 90 units/ml, more preferably less than about 80 units/ml, more preferably less than about 70 units/ml, more preferably less than about 63 units/ml, in some embodiments, less than about 42 units/ml, in some embodiments less than about 35 units/ml, and in some embodiments less than about 21 units/ml, an effective regimen of an anti-FRA therapeutic agent is administered to the subject.

In the methods of treatment described herein, CA125 expression level may be determined by any means known in the art, as described in paragraphs 0071 to 0078, supra.

Some embodiments of the methods of treating a subject with folate receptor alpha (FRA)-expressing ovarian cancer with an anti-FRA therapeutic agent described herein further involve determining the level of FRA in a sample derived from the subject; wherein an increase in the level of FRA in the sample derived from said subject as compared to the level of FRA in the control sample is indicative that the subject would benefit from treatment with an anti-FRA therapeutic agent. The level of FRA in the sample derived from the subject may be assessed as described in paragraphs 0079 through 0088, supra.

In some embodiments of the herein described methods of treatment, a baseline serum albumin (SA) concentration of the subject is determined. Methods for determining serum albumin (SA) concentration are known in the art. A baseline SA concentration of at least about 2.0 g/dL, preferably at least about 3.0 g/dL, and even more preferably at least about 3.2 g/dL is further indicative of a positive therapeutic response to the anti-FRA therapeutic agent. The baseline level of SA may be either a measurement of the SA level in the subject at a single timepoint or may involve measurement of SA levels in the subject at at least two points in time. Determination of the baseline level of SA in the subject may be performed upon diagnosis, upon surgical resection, upon initiation of first-line therapy, upon completion of first-line therapy, upon symptomatic progression, serologic progression, and/or radiologic progression of the cancer, upon initiation of second-line therapy, and/or upon completion of second-line therapy.

In some embodiments of the methods of treatment described herein, the anti-FRA therapeutic agent is an antibody that specifically binds to folate receptor alpha, preferably to FRA expressed on ovarian cancer cells; antigen-binding fragments of such an antibody; derivatives; and variants thereof. An exemplary antibody that specifically binds to folate receptor alpha may be an antibody selected from the group consisting of:
(c) an antibody comprising SEQ ID NO:1 as CDRH1, SEQ ID NO:2 as CDRH2, SEQ ID NO:3 as CDRH3, SEQ ID NO:4 as CDRL1, SEQ ID NO:5 as CDRL2 and SEQ ID NO:6 as CDRL3; or
(d) an antibody that binds the same epitope as farletuzumab.

In some embodiments, the antibody that specifically binds to folate receptor alpha comprises a mature light chain variable region comprising the amino acid sequence of SEQ ID NO:7 and/or a mature heavy chain variable region comprising the amino acid sequence of SEQ ID NO: 8. In preferred embodiments of the methods of treatment described herein, the anti-FRA therapeutic agent is farletuzumab. As described supra, other useful antibodies that specifically bind to folate receptor alpha comprise mature light and heavy chain variable regions having at least 90% and preferably at least 95% or 99% sequence identity to SEQ ID NO: 7 and SEQ ID NO: 8, respectively. Other useful anti-folate receptor alpha antibodies or derivatives thereof can competitively inhibit binding of farletuzumab to folate receptor alpha, as determined, for example, by immunoassay. A derivative of an anti-folate receptor alpha antibody can also be used in the practice of present methods. Typical modifications include, e.g., glycosylation, deglycosylation, acetylation, pegylation, phosphorylation, amidation, derivatization by known protecting/blocking groups, proteolytic cleavage, linkage to a cellular ligand or other protein, and the like. Additionally, the derivative may contain one or more non-classical amino acids.

In some embodiments of the methods of treatment provided herein, the anti-FRA therapeutic agent is administered to the subject to achieve a minimum serum concentration of at least about 50 μg/ml, preferably at least about 55 μg/ml, more preferably at least about 57.6 μg/ml, more preferably at least about 60 μg/ml, more preferably at least about 70 μg/ml, even more preferably at least about 80 μg/ml, and most preferably at least about 88.8 μg/ml, within about three weeks, preferably within about two weeks, and more preferably within about one week of administration of the initial dose of the anti-FRA therapeutic agent to the subject. In preferred embodiments, once such minimum serum concentration is achieved in a subject, the subject's serum level of the anti-FRA therapeutic agent remains above the Cmin or Ctrough for the remainder of therapy with the anti-FRA therapeutic agent.

Serum anti-FRA therapeutic agent concentration in the subject may be determined in the methods of treatment provided herein. In preferred embodiments, a minimum serum concentration of at least about 50 μg/ml, preferably at least about 55 μg/ml, more preferably at least about 57.6 μg/ml, more preferably at least about 60 μg/ml, more preferably at least about 70 μg/ml, even more preferably at least about 80 μg/ml, and most preferably at least about 88.8 μg/ml, within about three weeks, preferably within about two weeks, and more preferably within about one week of administration of the initial dose of the anti-FRA therapeutic agent to the subject, is indicative of a positive therapeutic response to the anti-FRA therapeutic agent.

In some embodiments of the methods of treatment provided herein, the anti-FRA therapeutic agent average area under the curve (AUC) pharmacokinetic (PK) exposure level is determined. For example, when the anti-FRA therapeutic agent is farletuzumab, farletuzumab average AUC PK exposure level is determined. An anti-FRA therapeutic agent average AUC PK exposure level of about 10 mg·h/ml or more, more preferably at least about 15 mg·h/ml or more, more preferably about 15.22 mg·h/ml or more, more preferably about 20 mg·h/ml or more, and even more preferably about 22.2 mg·h/L or more, is indicative of a positive therapeutic response to the anti-FRA therapeutic agent.

The present methods can be combined with other means of treatment such as surgery (e.g., debulking surgery), radiation, targeted therapy, chemotherapy, immunotherapy, use of growth factor inhibitors, or anti-angiogenesis factors. An anti-folate receptor alpha antibody or antigen-binding fragment thereof can be administered concurrently to a patient undergoing surgery, chemotherapy or radiation therapy treatments. Alternatively, a patient can undergo surgery, chemotherapy or radiation therapy prior or subsequent to administration of the anti-FRA therapeutic agent by at least an hour and up to several months, for example at least an hour, five hours, 12 hours, a day, a week, a month, or three months, prior or subsequent to administration of the anti-FRA therapeutic agent. For example, some embodiments of the methods of treatment provided herein further involve administration of a therapeutically effective amount of a platinum-containing compound and/or a taxane to the subject in addition to the anti-FRA therapeutic agent. Exemplary platinum-containing compounds are cisplatin or carboplatin. Examples of taxanes for use in the methods of treatment include but are not limited to paclitaxel, docetaxel, and semi-synthetic, synthetic, and/or modified versions and formulations thereof, including but not limited to nab-paclitaxel (Abraxane®), cabazitaxel (Jevtana®), DJ-927 (Tesetaxel®), paclitaxel poliglumex (Opaxio®), XRP9881 (Larotaxel®), EndoTAG+paclitaxel (EndoTAG®-1), Polymeric-micellar paclitaxel (Genexol-PM®), DHA-paclitaxel (Taxoprexin®), BMS-184476. The platinum-containing compound may be administered to the subject once every week, once every two weeks, once every three weeks, or once every four weeks. The taxane may be administered to the subject once every week, once every two weeks, once every three weeks, or once every four weeks. In embodiments in which both a taxane and a platinum-containing compound are administered to the subject as part of the treatment regimen, the taxane may be administered before, after, or simultaneously with the platinum-containing compound.

In some embodiments of the methods of treatment described herein, the subject may have received surgical resection of the ovarian cancer, first-line platinum-based therapy, first-line taxane-based therapy, and/or first-line platinum and taxane-based therapy for treatment of the ovarian cancer prior to determining the baseline level of CA125. In some embodiments of the methods in which the subject received surgical resection of the ovarian cancer, first-line platinum-based therapy, first-line taxane-based therapy, and/or first-line platinum and taxane-based therapy for treatment of the ovarian cancer prior to determining the baseline level of CA125, the subject may have exhibited symptomatic progression, serologic progression, and/or radiologic progression of the ovarian cancer prior to the step of determining the baseline level of CA125.

Administration of the therapeutic agents (including the anti-FRA therapeutic agent, the taxane, and/or the platinum-containing compound) in accordance with the methods of treatment described herein may be by any means known in the art.

Various delivery systems can be used to administer the therapeutic agents (including the anti-FRA therapeutic agent, the taxane, and/or the platinum-containing compound) including intradermal, intramuscular, intraperitoneal, intravenous, subcutaneous, intranasal, epidural, and oral routes. The agents can be administered, for example by infusion or bolus injection, by absorption through epithelial or mucocutaneous linings (e.g., oral mucosa, rectal and intestinal mucosa, and the like). Administration can be systemic or local.

The therapeutic agents can be administered by injection, by means of a catheter, by means of a suppository, or by means of an implant, the implant being of a porous, non-porous, or gelatinous material, including a membrane, such as a sialastic membrane, or a fiber. The therapeutic agents and pharmaceutical compositions thereof for use as described herein may be administered orally in any acceptable dosage form such as capsules, tablets, aqueous suspensions, solutions or the like.

Preferred methods of administration of the therapeutic agents include but are not limited to intravenous injection and intraperitoneal administration.

Alternatively, the therapeutic agents can be delivered in a controlled release system. For example, a pump can be used (see Langer, 1990, Science 249:1527-1533; Sefton, 1989, CRC Crit. Ref Biomed. Eng. 14:201; Buchwald et al., 1980, Surgery 88:507; Saudek et al., 1989, N. Engl. J. Med. 321:574). Alternatively, polymeric materials can be used (see Medical Applications of Controlled Release (Langer & Wise eds., CRC Press, Boca Raton, Fla., 1974); Controlled Drug Bioavailability, Drug Product Design and Performance (Smolen & Ball eds., Wiley, New York, 1984); Ranger & Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105.) Other controlled release systems are discussed, for example, in Langer, supra.

The therapeutic agents can be administered as pharmaceutical compositions comprising a therapeutically or prophylactically effective amount of the therapeutic agent(s) and one or more pharmaceutically acceptable or compatible ingredients. For example, the pharmaceutical composition typically includes one or more pharmaceutical carriers (e.g., sterile liquids, such as water and oils, including those of petroleum, animal, vegetable or synthetic origin, such as peanut oil, soybean oil, mineral oil, sesame oil and the like). Water is a more typical carrier when the pharmaceutical composition is administered intravenously. Saline solutions (e.g., phosphate buffered saline) and aqueous dextrose and glycerol solutions can also be employed as liquid carriers, particularly for injectable solutions. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, pH buffering agents (e.g., amino acids) and/or solubilizing or stabilizing agents (e.g., nonionic surfactants such as tween or sugars such as sucrose, trehalose or the like). The preferred formulation of farletuzumab contains farletuzumab, sodium phosphate, sodium chloride (NaCl), and polysorbate-80, pH 7.2. A preferred final formulation of farletuzumab contains 5 mg/mL farletuzumab, 10 mM sodium phosphate, 150 mM NaCl, and 0.01% polysorbate-80, pH 7.2.

The pharmaceutical compositions provided herein can take the form of solutions, suspensions, emulsion, tablets, pills, capsules, powders, sustained-release formulations and the like. Also included are solid form preparations which are intended to be converted, shortly before use, to liquid preparations. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the nucleic acid or protein, typically in purified form, together with a suitable amount of carrier so as to provide the form for proper administration to the patient. The formulations correspond to the mode of administration.

Typically, compositions for intravenous administration are solutions in sterile isotonic aqueous buffer. When necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or a concentrate in a hermetically scaled container such as an ampoule or sachette indicating the quantity of active agent. When the pharmaceutical composition is to be administered by infusion, it can be dispensed with an infusion bottle containing sterile pharmaceutical grade water or saline. When the pharmaceutical composition is administered by injection, an ampoule of sterile water for injection or saline can be provided so that the ingredients can be mixed prior to administration.

The amount of the therapeutic agent that is effective in the treatment or prophylaxis of ovarian cancer can be determined by standard clinical techniques. In addition, in vitro assays may optionally be employed to help identify optimal dosage ranges. The precise dose to be employed in the formulation also depends on the route of administration, and the stage of the cancer, and should be decided according to the judgment of the practitioner and each patient's circumstances. Effective doses may be extrapolated from dose-response curves derived from in vitro or animal model test systems. A dose can be formulated in animal models to achieve a circulating plasma concentration range that includes the IC₅₀ (i.e., the concentration of the test compound that achieves a half-maximal inhibition of symptoms) as determined in cell culture.

For example, toxicity and therapeutic efficacy of the agents can be determined in cell cultures or experimental animals by standard pharmaceutical procedures for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Agents that exhibit large therapeutic indices are preferred. When an agent exhibits toxic side effects, a delivery system that targets the agent to the site of affected tissue can be used to minimize potential damage to non-folate receptor alpha-expressing cells and, thereby, reduce side effects.

In some embodiments, the subject can be administered a therapeutic agent described herein in a daily dose range of about 0.01 μg to about 500 mg per kg of the weight of the subject. Typically, the dosage of the therapeutic agent (e.g., the anti-FRA therapeutic agent, preferably farletuzumab) administered to a patient with a folate receptor alpha-expressing ovarian cancer is about 0.1 mg/kg to about 100 mg/kg of the subject's body weight. More typically, the dosage administered to a subject is about 1.25 mg/kg to about 12.5 mg/kg of the subject's body weight, or even more typically about 2.5 mg/kg to about 10.0 mg/kg of the subject's body weight. In some embodiments, the dosage of the anti-FRA therapeutic agent, preferably farletuzumab, administered to a subject having folate receptor alpha-expressing ovarian cancer is about 5.0 mg/kg to about 7.5 mg/kg of the subject's body weight. In some embodiments of the methods of treatment described herein, a loading dose of the anti-FRA therapeutic agent of about 7.5 mg/kg to about 12.5 mg/kg, preferably about 10 mg/kg, is administered to the subject. In some embodiments of the methods of treatment described herein, two loading doses of the anti-FRA therapeutic agent of about 7.5 mg/kg to about 12.5 mg/kg weekly, preferably about 10 mg/kg, is administered to the subject in the first two weeks of treatment. In some embodiments, the dosage of the taxane administered to a subject having folate receptor alpha-expressing ovarian cancer is about 50 mg/m² to about 250 mg/m² of the subject's body weight, preferably about 75 mg/m² to about 200 mg/m². In some embodiments, the dosage of the platinum-containing compound administered to a subject having folate receptor alpha-expressing ovarian cancer is about AUC 3, preferably about AUC 4, more preferably about AUC 5-6. In a preferred embodiment, the subject is administered 10 mg/kg loading doses of farletuzumab for the first two weeks of treatment followed by 5 mg/kg farletuzumab intravenously weekly, carboplatin (about AUC 5-6) every three weeks, and taxane (paclitaxel (175 mg/m²) or docetaxel (75 mg/m²)) every three weeks. In a preferred embodiment, the subject receives 10 mg/kg loading doses of farletuzumab intravenously for the first two weeks of treatment followed by 5 mg/kg farletuzumab intravenously weekly, carboplatin (about AUC 5-6) intravenously every three weeks, and taxane (paclitaxel (175 mg/m²) or docetaxel (75 mg/m²)) intravenously every three weeks. In a preferred embodiment, at least six cycles of carboplatin and taxane are administered to the subject in combination with the weekly farletuzumab administration.

For effective treatment, one skilled in the art may recommend a dosage schedule and dosage amount of the therapeutic agent(s) adequate for the subject being treated. It may be preferred that dosing occur one to four or more times daily, once per week, once per every two weeks, once per every three weeks, or once per every four weeks for as long as needed. Typically, the anti-FRA therapeutic agent is administered to the subject weekly. In some preferred embodiments, the platinum-containing compound and/or taxane are administered to the subject once every week, once every two weeks, once every three weeks, or once every four weeks. The taxane may be administered to the subject once every week, once every two weeks, once every three weeks, or once every four weeks. In embodiments in which both a taxane and a platinum-containing compound are administered to the subject as part of the treatment regimen, the taxane may be administered before, after, or simultaneously with the platinum-containing compound.

The dosing may occur less frequently if the compositions are formulated in sustained delivery vehicles. The dosage schedule may also vary depending on the active drug concentration, which may depend on the needs of the subject

Kits

Further provided herein are kits for identifying a subject having ovarian cancer that will be responsive to treatment with an anti-folate receptor alpha (FRA) therapeutic agent. In some embodiments, the kits contain an anti-CA125 antibody, a vessel for containing the antibody when not in use, and instructions for using the anti-CA125 antibody for determining the level of CA125 of a subject. The instructions may specify that a baseline CA125 level is less than about eight times the upper limit of normal (ULN) for CA125, preferably less than about seven times the ULN for CA125, more preferably less than about six times the ULN for CA125, more preferably less than about five times the ULN for CA125, more preferably less than about four times the ULN for CA125, more preferably less than about three times the ULN for CA125, more preferably less than about two times the ULN for CA125 and, in some embodiments, less than about the ULN for CA125, is indicative of a subject who would benefit from treatment with an anti-FRA therapeutic agent. Alternatively, the instructions may specify that a baseline CA125 level that is less than about 164 units/ml, preferably less than about 150 units/ml, more preferably less than about 140 units/ml, more preferably less than about 130 units/ml, more preferably less than about 120 units/ml, more preferably less than about 110 units/ml, more preferably less than about 100 units/ml, even more preferably less than about 90 units/ml, more preferably less than about 80 units/ml, more preferably less than about 70 units/ml, more preferably less than about 63 units/ml, more preferably less than about 42 units/ml, in some embodiments less than about 35 units/ml, and in some embodiments less than about 21 units/ml, is indicative of a subject who would benefit from treatment with an anti-FRA therapeutic agent. In some embodiments, the kits also contain an anti-FRA antibody, a vessel for containing the anti-FRA antibody when not in use, and instructions for using the anti-FRA antibody for determining the level of FRA of a subject. In some embodiments, the kits may contain an anti-serum albumin (SA) antibody, a vessel for containing the anti-SA antibody when not in use, and instructions for using the anti-SA antibody for determining the level of SA of a subject. One or more additional containers may enclose elements, such as reagents or buffers, to be used in the molecular marker assay(s). Such kits can also, or alternatively, contain a detection reagent that contains a reporter group suitable for direct or indirect detection of antibody binding.

Also provided herein are kits for treating a subject having ovarian cancer that will be responsive to treatment with an anti-FRA therapeutic agent comprising the anti-FRA therapeutic agent, a vessel for containing the anti-FRA therapeutic agent when not in use, and instructions for use of the anti-FRA therapeutic agent Farletuzumab is the preferred anti-FRA therapeutic agent in the kits. The instructions may specify that a baseline CA125 level is less than about eight times the upper limit of normal (ULN) for CA125, preferably less than about seven times the ULN for CA125, more preferably less than about six times the ULN for CA125, more preferably less than about five times the ULN for CA125, more preferably less than about four times the ULN for CA125, more preferably less than about three times the ULN for CA125, more preferably less than about two times the ULN for CA125, and, in some embodiments, less than about the ULN for CA125, is indicative of a subject who would benefit from treatment with an anti-FRA therapeutic agent. Alternatively, the instructions may specify that a baseline CA125 level that is less than about 164 units/ml, preferably less than about 150 units/ml, more preferably less than about 140 units/ml, more preferably less than about 130 units/ml, more preferably less than about 120 units/ml, more preferably less than about 110 units/ml, more preferably less than about 100 units/ml, even more preferably less than about 90 units/ml, more preferably less than about 80 units/ml, more preferably less than about 70 units/ml, more preferably less than about 63 units/ml, in some embodiments, less than about 42 units/ml, in some embodiments less than about 35 units/ml, and in some embodiments less than about 21 units/ml, is indicative of a subject who would benefit from treatment with an anti-FRA therapeutic agent. In some embodiments, the kits for treating a subject having ovarian cancer that will be responsive to treatment with an anti-FRA therapeutic agent also contain an anti-CA125 antibody, a vessel for containing the anti-CA125 antibody when not in use, and instructions for using the anti-CA125 antibody for determining a baseline level of CA125 in a biological sample obtained from the subject. In some embodiments, the kits also contain an anti-FRA antibody, a vessel for containing the anti-FRA antibody when not in use, and instructions for using the anti-FRA antibody for determining the level of FRA of a subject. In some embodiments, the kits may contain an anti-serum albumin (SA) antibody, a vessel for containing the anti-SA antibody when not in use, and instructions for using the anti-SA antibody for determining the level of SA of a subject.

The kits for treating a subject having ovarian cancer that will be responsive to treatment with an anti-FRA therapeutic agent also may contain additional therapeutic agents (e.g., a platinum-containing compound and/or a taxane) as described herein. Examples of platinum-containing compounds for inclusion in the kits include, but are not limited to, cisplatin and carboplatin. Examples of taxanes for inclusion in the kits include, but are not limited to, paclitaxel, docetaxel, and semi-synthetic, synthetic, and/or modified versions and formulations thereof, including but not limited to nab-paclitaxel (Abraxane®), cabazitaxel (Jevtana®), DJ-927 (Tesetaxel®), paclitaxel poliglumex (Opaxio®), XRP9881 (Larotaxel®), EndoTAG+paclitaxel (EndoTAG®-1), Polymeric-micellar paclitaxel (Genexol-PM®), DHA-paclitaxel (Taxoprexin®), BMS-184476. The therapeutic agents can be in any of a variety of forms suitable for distribution in a kit. Forms of the therapeutic agents suitable for distribution in the kits can include a liquid, powder, tablet, suspension and the like formulation for providing the therapeutic agent. The kits can also include a pharmaceutically acceptable diluent (e.g., sterile water) for injection, reconstitution or dilution of the therapeutic agent(s). One or more additional containers may enclose elements, such as reagents or buffers, to be used in the molecular marker assay(s). Such kits can also, or alternatively, contain a detection reagent that contains a reporter group suitable for direct or indirect detection of antibody binding.

Kits also typically contain a label or instructions for use in the methods described herein. The label or instruction refers to any written or recorded material that is attached to, or otherwise accompanies a kit at any time during its manufacture, transport, sale or use. It can be a notice in the form prescribed by a governmental agency regulating the manufacture, use or sale of pharmaceuticals or biological products, which notice reflects approval by the agency of manufacture, use or sale for human administration. The label or instruction can also encompass advertising leaflets and brochures, packaging materials, instructions, audio or videocassettes, computer discs, as well as writing imprinted directly on the pharmaceutical kits.

The following example is provided to further describe some of the embodiments disclosed herein. The example is intended to illustrate, not to limit, the disclosed embodiments.

### Example

### Multicenter, Double-Blind, Randomized (1:1:1 Ratio), Placebo-Controlled Trial of Two Dose Levels of Farletuzumab or Placebo Combined with Carboplatin and a Taxane

Subjects received farletuzumab (or matching placebo) once weekly throughout the study. Carboplatin/taxane were administered once every 3 weeks (I cycle) for 6 cycles.

Additional cycles were to be administered at the investigator's discretion. A drug-drug interaction (DDI) substudy was conducted to determine whether a pharmacokinetic interaction exists between farletuzumab and carboplatin, paclitaxel, or docetaxel. Single-agent test drug was to be administered weekly after discontinuation of chemotherapy, until disease progression as defined by modified RECIST criteria. During the follow-up period, survival status and additional therapy for ovarian cancer were captured until death or study termination by the sponsor.

Number of Subjects (Planned and Enrolled)

A total of 1080 subjects were planned; 1100 were enrolled and randomly assigned to treatment with carboplatin/taxane and either one of two double-blind farletuzumab dose levels (1.25 or 2.5 mg/kg) or placebo in a 1:1:1 ratio. Randomization was stratified by (1) length of first remission, (2) route of administration for first-line therapy (intraperitoneal [i.p.] versus intravenous [i.v.]), (3) planned taxane therapy, and (4) geographic region (North America and Western Europe versus Other Participating Countries).

| | Placebo + | FAR 1.25 mg/kg + | FAR 2.5 mg/kg + |
|---|---|---|---|
| | Carboplatin/Taxane | Carboplatin/Taxane | Carboplatin/Taxane |
| Analysis Population | n (%) | n (%) | n (%) |
| Intent-to-Treat (ITT) | 364 (100) | 370 (100) | 366 (100) |
| Safety* | 352 (96.7) | 376 (101.6) | 363 (99.2) |
| Per Protocol | 332 (91.2) | 348 (94.1) | 342 (93.4) |
| Combination Therapy | 352 (96.7) | 376 (101.6) | 363 (99.2) |
| Single-Agent Maintenance | 252 (69.2) | 272 (73.5) | 255 (69.7) |
| Tumor Response Evaluable | 331 (90.9) | 350 (94.6) | 328 (89.6) |
| (based on Independent | | | |
| Assessment) | | | |
| Serologic Response Evaluable | 272 (74.7) | 272 (73.5) | 273 (74.6) |

*Nine subjects randomly assigned to treatment (three in each of the three treatment groups) did not receive study medication. In addition, nine subjects who were randomized to placebo received at least one dose of farletuzumab; these subjects are counted in the FAR 1.25 mg/kg + carboplatin/taxane treatment group.

Diagnosis and Main Criteria for Inclusion

Subjects had platinum-sensitive ovarian cancer treated initially with surgery and which had responded to first-line platinum and taxane-based chemotherapy followed by relapse between 6 and 24 months from the time of completion of first-line therapy, as defined by the presence of measurable disease.

Test Treatment, Dose, Mode of Administration, and Batch Numbers

Farletuzumab was supplied by the sponsor as a solution for i.v. injection, 5 mg/mL, 5 mL per vial. Normal saline was used as placebo and was supplied by the investigative site unless prohibited by local regulations or institutional policy. Farletuzumab batch numbers were A46930, A58005B, A58028, A62367, A62367B, W0004711, W0004714, W0004852, W0004996, W0004997, W0004998, W0005435, W0005436, W0005673, W0005715, W0005735, and W0006012.

Reference Therapy, Dose, Mode of Administration, and Batch Numbers

Carboplatin (AUC 5-6), paclitaxel (175 mg/m2), and docetaxel (75 mg/m2) for i.v. use were supplied by the investigative site unless prohibited by local regulations or institutional policy.

Duration of Treatment

Subjects could continue to receive treatment until their disease progressed or they experienced unacceptable toxicity or intercurrent illness that prevented further administration of study medication, the subject or physician requested discontinuation, or changes in the subject's condition rendered the subject unacceptable for further treatment in the judgment of the investigator.

Assessments

Efficacy

Computerized tomography (CT) scans or magnetic resonance imaging (MRI) were performed every 6 weeks (every second cycle) during combination therapy, and every 9 weeks (every third cycle) during maintenance therapy. Blood was drawn to determine CA125 levels every 3 weeks during combination therapy and every 9 weeks (every third cycle) during maintenance therapy. Historical CA125 serum levels were obtained when available. CA125 serum levels were assessed by Immulite® assay (Siemens Healthcare).

Pharmacokinetics

Blood was drawn at Cycle 2 for measurement of serum levels of farletuzumab and chemotherapeutic agents. Additional blood was drawn at a single time point during administration of single agent test drug (farletuzumab or placebo) at least 3 weeks after discontinuation of chemotherapy.

Effects of farletuzumab on pharmacokinetics for carboplatin, paclitaxel, or docetaxel were analyzed primarily via noncompartmental analysis for subjects in the substudy. Effects of concomitant chemotherapeutic agents on farletuzumab pharmacokinetics was assessed by population pharmacokinetic (PPK) analysis using Nonlinear Mixed Effect Modeling (NONMEM), after combining all farletuzumab PK data from other farletuzumab clinical studies.

PK/PD PFS analysis data was available from 1081 subjects from the Phase 3 study, of whom 729 received farletuzumab and 352 received placebo. Model based analyses consisted of a population PK model for farletuzumab, population PK/PD models for longitudinal tumor size measurement, and PFS data. All models except time-to event analysis for PFS were developed using NONMEM version 7.2 interfaced with PD×Pop 5.0. Time-to-event analysis for PFS was performed using TIBCO Spotfire S-plus 8.1. Model building and covariate assessments were conducted using standard methods in accordance with regulatory guidelines.

The final population PK model was used to derive individual PK parameters and farletuzumab exposures, which were then incorporated into the PK/PD datasets to be used in the subsequent population PK/PD analyses. Time-to-event analysis for PFS was performed for study 004. PFS data was explored using Kaplan-Meier and Cox regression analyses using survfit( ) and coxph( ) functions, respectively in S-plus.

Pharmacogenomics/Pharmacogenetics

Archival tumor samples, peripheral blood mononuclear cells, and serum were collected and banked to support a retrospective analysis.

Safety

Evaluation of safety included review of clinical (adverse event [AE] reports, physical examination findings, vital sign measurements, electrocardiograms [ECGs], and Karnofsky Performance Status [KPS]) and laboratory data. Severity of AEs was graded using the National Cancer Institute Common Terminology Criteria for Adverse Events (NCI CTCAE, Version 3.0) classification.

Quality of life (QoL) was assessed using Functional Assessment of Cancer Therapy-Ovarian (FACT-O), v 4.0. Resource utilization was assessed through recorded hospitalizations, unscheduled office visits, and admissions to hospice or nursing home.

Statistical Methods

The primary endpoint of the study was PFS based on central, independent radiologic assessments using the modified RECIST criteria. For the primary efficacy analyses, multiplicity for the two comparisons of each of the farletuzumab dose groups versus control group in PFS was adjusted so that the study level type I error rate is controlled to be lower than 0.05 significance level (2-sided). The primary analysis population for all efficacy endpoints was the Intent-to-Treat Population (ITT), defined as all subjects assigned to treatment per IVRS/IWRS. Evaluable Populations were defined as all subjects who received at least one dose of study medication and who had a baseline and at least one on-treatment assessment performed, sufficient to assess the endpoint of interest. These populations were used to evaluate tumor response, farletuzumab serum drug levels, and subject-reported outcomes (QoL and resource utilization).

Progression-free survival was defined as the time (in months) from the date of randomization to the date of the first observation of progression based on the independent radiologic assessment (modified RECIST), or date of death, whatever the cause. The cut-off date for PFS was to be based on the observation of the 391st event in either the low-dose farletuzumab and placebo groups combined or the high-dose farletuzumab and placebo groups combined, whichever occurred later. Unblinded monitoring of the total number of events was to be performed by the independent Data Monitoring Committee (DMC), and the sponsor was to be notified when the required number of events (391) had been observed in both combinations (low-dose farletuzumab versus placebo plus high-dose farletuzumab versus placebo) for purposes of conducting the primary analysis. The cut off date for PFS was to be used for secondary efficacy variables, as well as survival data supporting the interim survival analysis. Overall survival (OS) was defined as the time from the date of randomization to the date of death, due to all causes.

Pairwise comparisons between the two farletuzumab dose groups and placebo were based on the stratified log-rank test (one-sided), based on the randomization strata. In addition, the hazard ratio (HR) was estimated based on Cox's proportional hazards model. Sensitivity analyses were performed using the unstratified log-rank test.

Quality of life was analyzed for treatment differences using a mixed model with repeated measures ANOVA for each functional domain of the FACT-O and the three composite measures: FACT-O TOI (Treatment Outcome Index), FACT-O, and FACT-G. Cycle effects and interactions between cycle and treatment were also tested. Two other statistical methods were applied: a Pattern Mixture Model and Generalized Estimating Equations with adjustments for baseline score, PFS status, geographic region, length of first remission, route of administration, and baseline Karnofsky Performance Status.

Sample size considerations are based on the primary PFS endpoint. The median PFS in the placebo group is hypothesized to be 12 months. A target HR (farletuzumab:placebo) of 0.70, equivalent to a 43% improvement in PFS, and a median PFS for subjects treated with farletuzumab of 17.14 months, are assumed for both the high-dose and low-dose groups. Under these assumptions, log-rank tests with an overall two sided type I error rate of 0.05 would have at least 95% power (see below) to claim at least one positive comparison for farletuzumab dose groups versus placebo when the target number of events (i.e., progressive disease or death) in either the low-dose farletuzumab and placebo groups combined or the high-dose farletuzumab and placebo groups combined is 391, whichever occurred later. The sample size calculations have accounted for a multiplicity adjustment for the two farletuzumab dose group comparisons versus placebo. The targeted number of 391 events for each pairwise treatment:control comparison was derived based on a log-rank test at the pairwise one-sided 0.0125 significance level with 90% power for a HR of 0.70.

Approximately 1080 subjects (360 in each of three groups) were to be randomly assigned to achieve the specified number of events. Study follow-up for survival was extended until the targeted number of events was reached to adequately power the study for overall survival (OS).

Two interim analyses were planned:

Serologic Response (CA125) Futility Analysis: An interim analysis to evaluate the futility of both dose groups of farletuzumab on the basis of serologic response (CA125) was conducted after approximately 300 subjects completed at least 3 months in the study; and

Interim Analysis for OS: One OS interim analysis to evaluate superiority (or inferiority) of the two farletuzumab dose groups to the placebo group was planned. This analysis was performed to accompany the primary analysis of the study based on PFS. Survival status reported up to the primary analysis cut-off date is included in this analysis.

Results

Subject Disposition/Analysis Sets

A total of 1217 subjects were screened for entry into the study. Of these subjects, 115 were screen failures, and 2 subjects were randomly assigned to test article in error.

The remaining 1100 subjects were randomly assigned to treatment, and comprised the ITT population. Of these, 9 subjects (3, in each treatment group) did not receive any study drug. Thus, a total of 1091 subjects (361 in the placebo+carboplatin/taxane group, 367 in the FAR 1.25 mg/kg+carboplatin/taxane group, and 363 in the FAR 2.5 mg/kg+carboplatin/taxane group) received at least one dose of study drug. Nine of the subjects who were assigned to the placebo+carboplatin/taxane group received the incorrect test article during the study period due to pharmacy errors; safety and exposure data for these subjects were analyzed according to the treatment received. Thus, the safety analysis set was comprised of 352 in the placebo+carboplatin/taxane group, 376 in the FAR 1.25 mg/kg+carboplatin/taxane group, and 363 in the FAR 2.5 mg/kg+carboplatin/taxane group.

Of the 1091 subjects who initiated combination therapy, 287 (26.3%) discontinued combination therapy. Overall, almost half of all treatment discontinuations were due to PD, either by radiologic assessment (42.9%) or by clinical assessment (2.8%). Other primary reasons for discontinuation from combination therapy were nonfatal AEs (15.0%), subject choice (12.9%), withdrawn consent (10.8%), investigator discretion (5.9%), and fatal AE (5.6%).

Of the 779 subjects who initiated single-agent maintenance therapy, 603 (77.4%) discontinued test article. As shown in Table 1, the most common primary reason for treatment discontinuation was PD by radiological assessment (82.8%) or by clinical assessment (5.6%). Other primary reasons for discontinuation from single-agent maintenance therapy included subject choice (5.3%), nonfatal AEs (2.3%), withdrawn consent (1.8%), and investigator discretion (1.8%).

TABLE 1

| | | FAR 1.25 | FAR 2.5 | | |
|---|---|---|---|---|---|
| | Placebo + | mg/kg + | mg/kg + | | |
| | Carboplatin/ | Carboplatin/ | Carboplatin/ | | Combined |
| | Taxane | Taxane | Taxane | Total FAR | Total |
| Parameter | (N = 364) | (N = 370) | (N = 366) | (N = 736) | (N = 1100) |
| Length of | | | | | |
| remission^{a} | | | | | |
| (from | | | | | |
| IVRS/IWRS), | | | | | |
| n (%) | | | | | |
| 6 to < 12 | 194 (53.3) | 196 (53.0) | 193 (52.7) | 389 (52.9) | 583 (53.0) |
| months | | | | | |
| 12 to < 18 | 108 (29.7) | 112 (30.3) | 111 (30.3) | 223 (30.3) | 331 (30.1) |
| months | | | | | |
| 18 to 24 | 62 (17.0) | 62 (16.8) | 62 (16.9) | 124 (16.8) | 186 (16.9) |
| months | | | | | |
| Route of | | | | | |
| administration | | | | | |
| for first-line | | | | | |
| therapy | | | | | |
| (from | | | | | |
| IVRS/IWRS), | | | | | |
| n (%) | | | | | |
| Intraperitoneal | 26 (7.1) | 28 (7.6) | 26 (7.1) | 54 (7.3) | 80 (7.3) |
| Intravenous | 338 (92.9) | 342 (92.4) | 340 (92.9) | 682 (92.7) | 1020 (92.7) |
| Planned | | | | | |
| taxane | | | | | |
| therapy | | | | | |
| (from | | | | | |
| IVRS/IWRS), | | | | | |
| n (%) | | | | | |
| Paclitaxel | 294 (80.8) | 298 (80.5) | 296 (80.9) | 594 (80.7) | 888 (80.7) |
| Docetaxel | 70 (19.2) | 72 (19.5) | 70 (19.1) | 142 (19.3) | 212 (19.3) |

Efficacy

As shown in Table 2, median PFS based on independent review in the ITT population ranged from 9.0 to 9.7 months and was not statistically significant between the FAR and placebo treatment groups (all subjects received active chemotherapy). Median OS in the ITT population ranged from 27.8 months to 29.5 months and was not statistically significant between FAR treatment groups and placebo. Median PFS based on serologic progression (CA125) was 12.0 months in the placebo group, 12.6 months in the FAR 1.25 mg/kg group, and could not be estimated in the FAR 2.5 mg/kg group. The P value (one-sided log rank test) for the difference between the FAR 2.5 mg/kg group and placebo was 0.0437 for the stratified analysis, ITT population, 0.0227 for the unstratified analysis, ITT population, and 0.0412 for the stratified analysis, Safety Analysis Set. Median PFS by GCIG criteria ranged from 8.4 months to 8.6 months and was not statistically significant between FAR treatment groups and placebo. An objective response rate (CR/PR) of 56% based on RECIST criteria (independent review) was observed in each treatment group, with no statistically significant differences between FAR treatment groups and placebo. Serologic response was normalized in 60% to 65% of subjects in each treatment group, with no statistical difference between groups. The FAR 2.5 mg/kg group consistently outperformed the FAR 1.25 mg/kg group with regard to PFS based on independent assessment, serologic criteria, or GCIG criteria, but did not reach clinical or statistical significance compared to the placebo group. Stratification factors (length of first remission, route of administration for first-line therapy, planned taxane therapy, and geographic region) were well balanced and did not appear to affect response.

TABLE 2
Efficacy Analysis of Primary and Secondary Endpoints

| Endpoint | Placebo | | 1.25 | mg/kg FAR | 2.5 | mg/kg FAR |
|---|---|---|---|---|---|---|
| PFS | 9.0 | mo | 9.6 | mo (0.99 HR) | 9.7 | mo (0.86 HR) |
| OS | 26.2 | mo | 26.6 | mo (1.07 HR) | 26.7 | (1.03 HR) |
| GCIG PFS | 8.4 | mo | 8.6 | mo (1.01 HR) | 8.7 | mo (0.87 HR) |

| | | | | | | |
|---|---|---|---|---|---|---|
| >2^{nd} vs. 1^{st} remission | 7 | (3.5%) | 7 | (3.5%) | 13 | (6.5%) |

| | | | |
|---|---|---|---|
| subjects | | | |
| Response Rate | 59.5% | 58.6% | 62.2% |
| Clinical Benefit | 68.0% | 67.4% | 68.0% |

| | | | |
|---|---|---|---|
| Serologic PFS | 12.0 | mo | Combined FAR 13.8 mo (0.85 HR) |

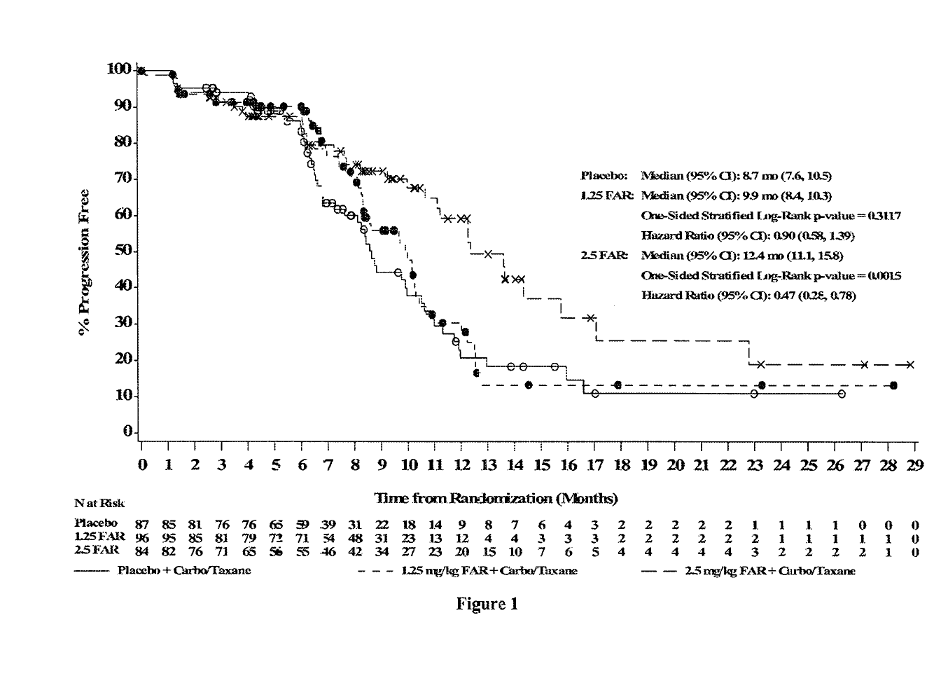

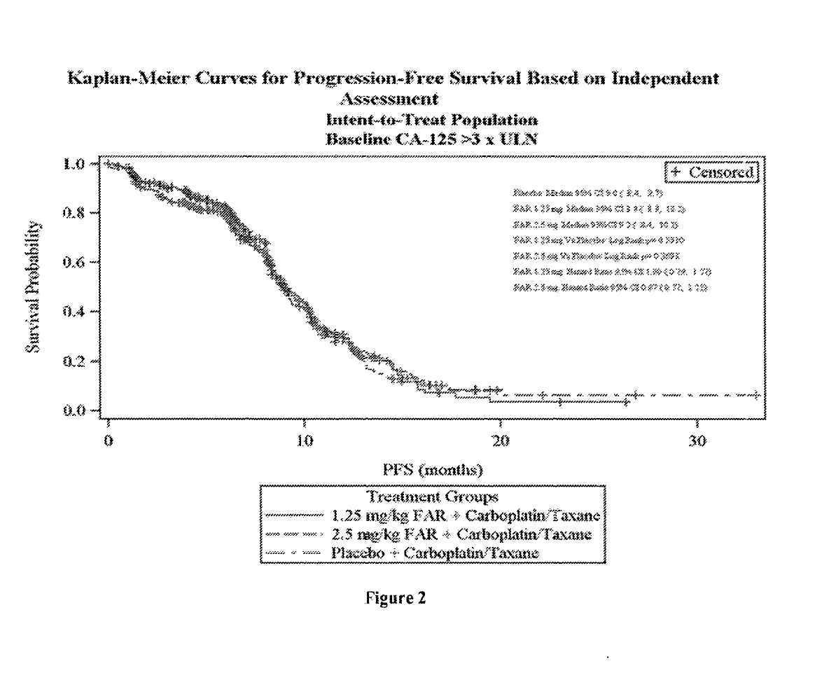

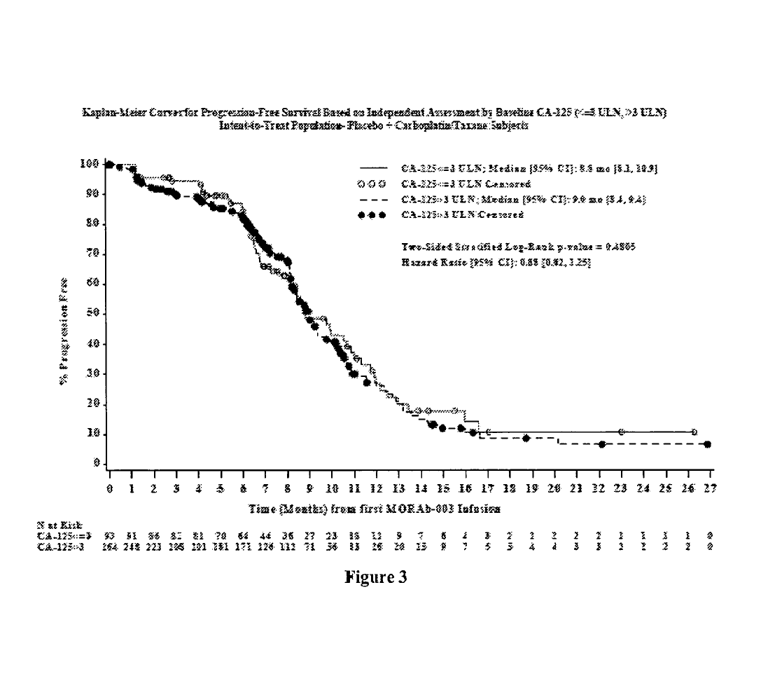

For the FAR 2.5 mg/kg group, baseline CA125 levels ≦3× the upper limit of normal (ULN) appeared to correlate with longer PFS and OS. Compare, for example, FIG. 1 to FIG. 2. FIG. 1 shows CA125 effect on median progression-free survival (PFS) of patients having a baseline CA125 serum concentration three times ULN (3×ULN=63 U/ml) or less. In this biomarker subgroup, patients receiving the high dose of farletuzumab (2.5 mg/kg) have a statistically significant difference in median PFS of 13.6 months compared to 8.8 months in placebo (HR=0.49; p=0.0014). Solid line/open circle represents results for the group that received placebo+carboplatin/Taxane. Dotted line, closed circle represents results for treatment group that received 1.25 mg/kg FAR+Carboplatin/Taxane. Dotted line, X represents results for the treatment group that received 2.5 mg/kg FAR+Carboplatin/Taxane. FIG. 2 shows CA125 effect on median progression-free survival (PFS) of patients having a baseline CA125 serum concentration greater than three times ULN (63 U/ml). Median PFS was 9 months in placebo and 8.8 months in both farletuzumab low and high doses. Therefore, farletuzumab did not appear to have a positive effect on PFS based on a patient subgroup with higher levels of CA125. FIG. 3 provides a Kaplan-Meier curve comparing PFS in placebo patients by baseline 3×ULN CA125 level. 93 of 357 total placebo patients had a CA125<3×ULN, with a median PFS of 8.8 months compared to 9.0 months in the >3×ULN patients. The median PFS is similar and there is not a statistically significant difference between the two groups (HR=0.88; p=0.48). Therefore, baseline CA125 in patients who received placebo combined with standard of care chemotherapy did not have any statistical or clinical difference in median PFS, where CA125 did not indicate any prognostic or predictive effect in this patient population.

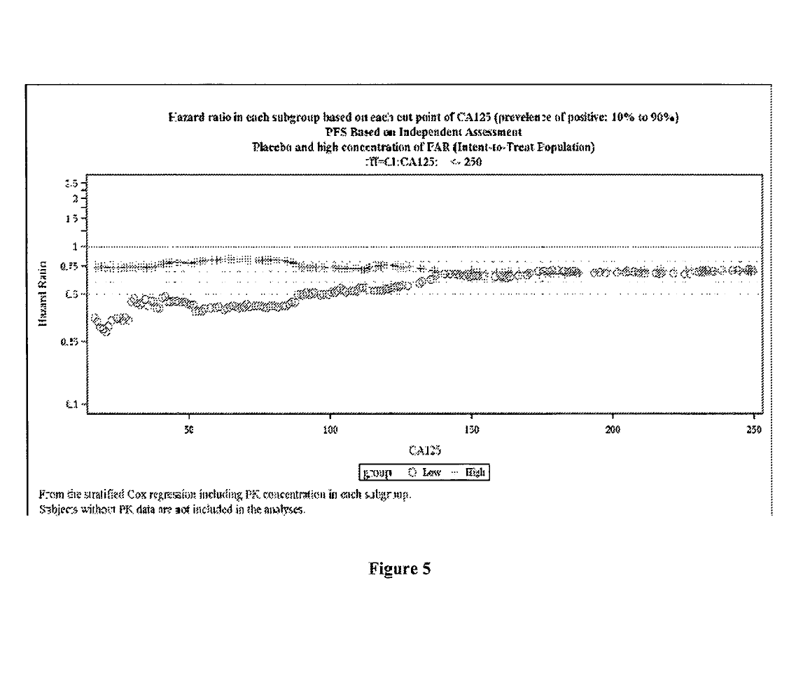

FIG. 5 illustrates the optimization of clinical effects of farletuzumab as measured by progression-free survival (PFS) versus CA125 levels. A threshold of three times the CA125 ULN was prespecified in analysis plans to identify differences between levels of elevated CA125, and demonstrated a positive effect for the lower CA125 subgroup. Accordingly, additional analysis has demonstrated additional potential cutpoint values that could be used to optimize a CA125 value cutpoint that maximize the treatment effect in the largest subgroup possible. FIG. 5 graphs hazard ratios for CA125 at CA125 cutpoint values from 0-250 in patients with high median pharmacokinetic (PK) exposure levels independent of farletuzumab dose. The lower curve (blue circles) indicates hazard ratios for subjects at or below the CA125 value for that estimate, while the higher curve (red crosses) illustrates the hazard ratios for those subjects above that same cutpoint. As shown, a robust clinical effect is observed in patients with high farletuzumab PK exposure levels exhibiting about 130 U/ml or less of CA125, with a hazard ratio of approximately 0.5 or better up to this value.

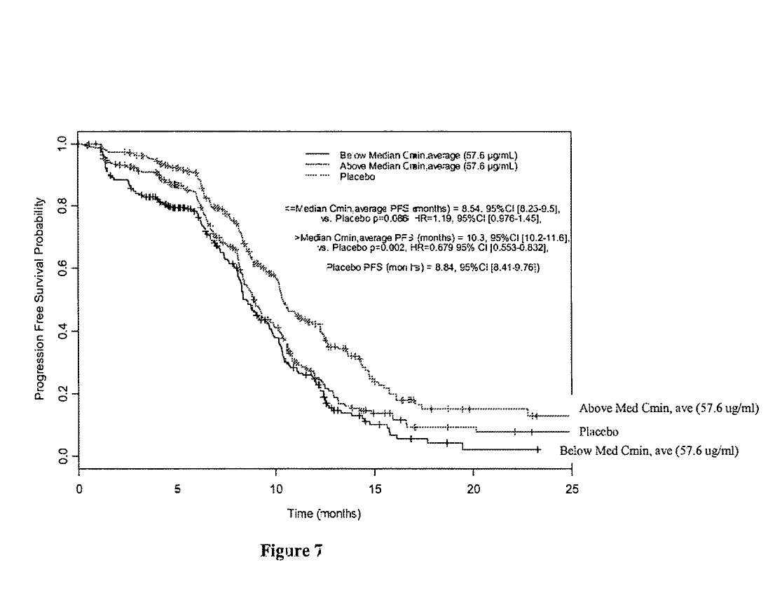

When compared to placebo and to lower antibody concentrations (based on their trough level or lowest sampling point, not dose treatment), patients with higher farletuzumab concentration levels have a statistically significant difference in median PFS (10.3 vs 8.5 months). FIG. 7 illustrates median progression-free survival (PFS) for patients based on Cmin farletuzumab pharmacokinetic exposure levels. Kaplan Meier curves for PFS were developed demonstrating a difference in PFS by median average Cmin or lowest point PK trough levels independent of the assigned farletuzumab dose. PFS in subjects with farletuzumab Cmin concentrations above median levels (>57.6 μg/mL) showed a statistically significant improvement in PFS when compared to placebo (p=0.002, HR=0.679, 95% CI [0.553-0.832]). Patients in the higher average farletuzumab Cmin had an average PFS of 10.3 months (higher plotted curve). Patients with a higher average farletuzumab Cmin level had better PFS than those patients with placebo and lower average Cmin, indicating an exposure response relationship.

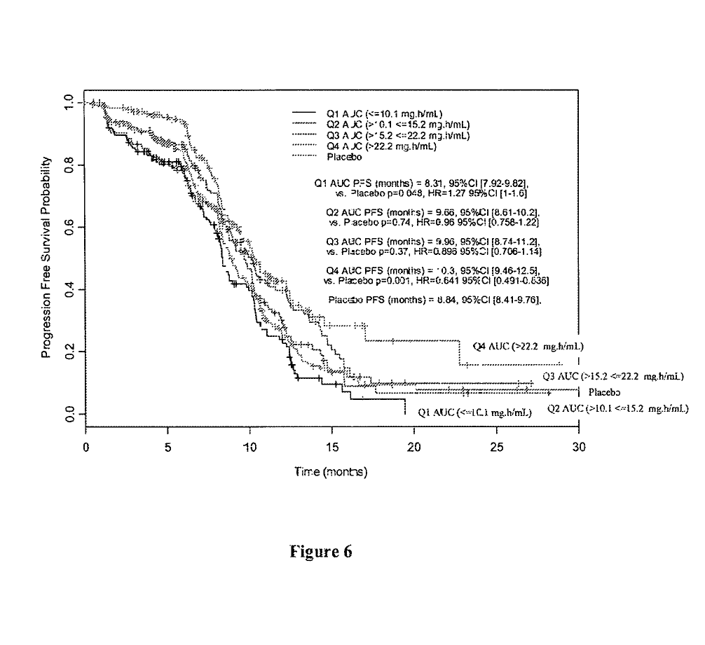

Similar analysis was done based on area under the curve (AUC) pharmacokinetic levels to assess exposure levels over time, and a consistent result was found where patients achieving the highest quartile AUC had a higher PFS when compared to other quartiles, with a median AUC 4th quartile PFS of 10.3 months versus 8.8 in placebo. FIG. 6 illustrates progression-free survival by quartile of farletuzumab average area under the curve (AUC) pharmacokinetic exposure levels. Kaplan-Meier plots for subjects with farletuzumab average AUC pharmacokinetic exposure levels above median levels (>15.22 mg·h/mL) and in particular for the upper quartile (Q4>22.8 mg·h/mL) showed a significant relationship for PFS in comparison to placebo (p=0.001, HR=0.641, 95% CI [0.491-0.836]). PFS for those subjects with farletuzumab in Q4 (>22.2 mg·h/L) had a longer PFS when compared to other lower AUC quartiles, and the overall Q4 PFS was 10.3 months compared to 8.84 months in placebo.

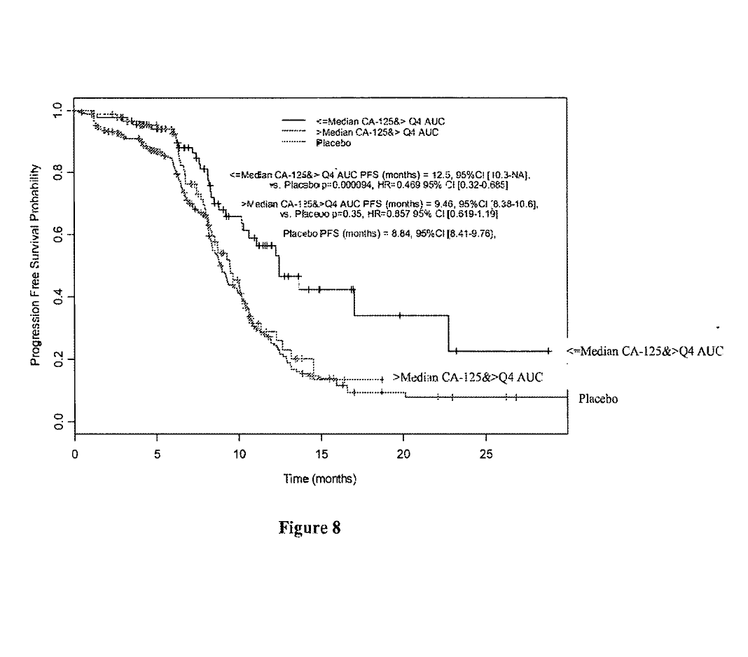

FIG. 8 further illustrates a Kaplan-Meier curve for PFS comparing median CA125 levels and placebo in the farletuzumab highest concentration population. Patients in the highest 75% quartile concentration level by AUC (Q4) are divided above or below the median CA125 value (164 IU/ml). Those Q4 AUC concentration patients with a CA125 below the median have a statistically significant difference in PFS of 12.5 months versus 8.84 in placebo (HR=0.46; p=0.000094). Patients with this same higher Q4 AUC level that have a higher than median CA125 only have an improvement of PFS of 9.46 months which is not statistically significant.

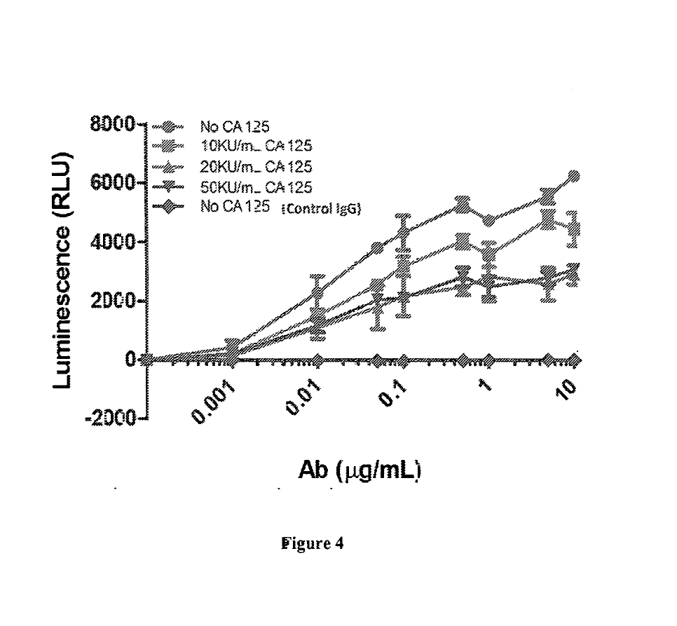

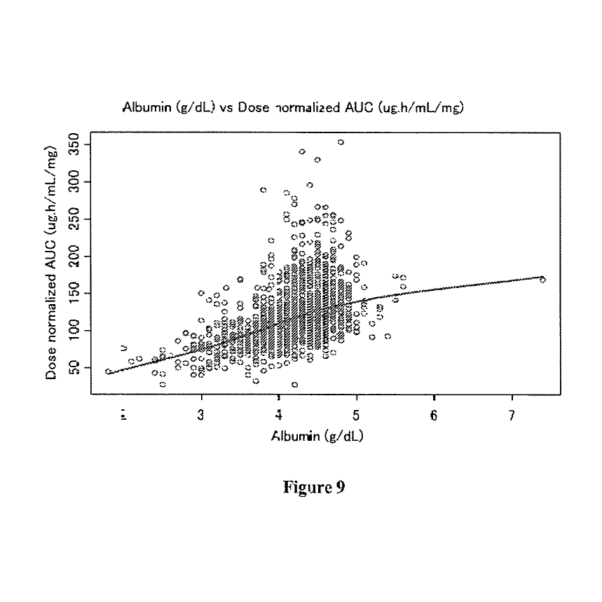

Analyses have focused on factors that may influence antibody concentration levels for patients that cannot retain adequate exposure levels and could therefore be excluded or identified prior to treatment. Baseline albumin was one parameter indicated in the pharmacokinetic analysis that correlates with farletuzumab exposure levels. Lower levels of baseline albumin correlated with lower farletuzumab AUC levels, and baseline albumin below the normal limits was associated with farletuzumab AUC levels below those indicated necessary for the exposure-response relationship. FIG. 9 illustrates the relationship between farletuzumab exposure and patient albumin levels. In the population pharmacokinetic analysis, farletuzumab clearance was identified to decline with increasing baseline albumin levels. Lower baseline albumin is associated with a decrease in farletuzumab dose-normalized concentration exposure (AUC) levels. In addition, FIG. 4 illustrates the dose-dependent inhibition of farletuzumab cytotoxicity via ADCC by CA125. Antibodies (Farletuzumab or negative control IgG), effector cells, and increasing concentrations of CA125 were added to human FRA-expressing Chinese hamster ovary (CHO-hFR-α) target cells. Increasing luminescence indicates effector cell activation (ADCC activity) as described by Promega ADCC Reporter Bioassay Core Kit. As shown in the figure, there was a dose-dependent inhibition of Farletuzumab ADCC activity with increasing levels of CA125, with a maximal inhibition of approximately 50%.

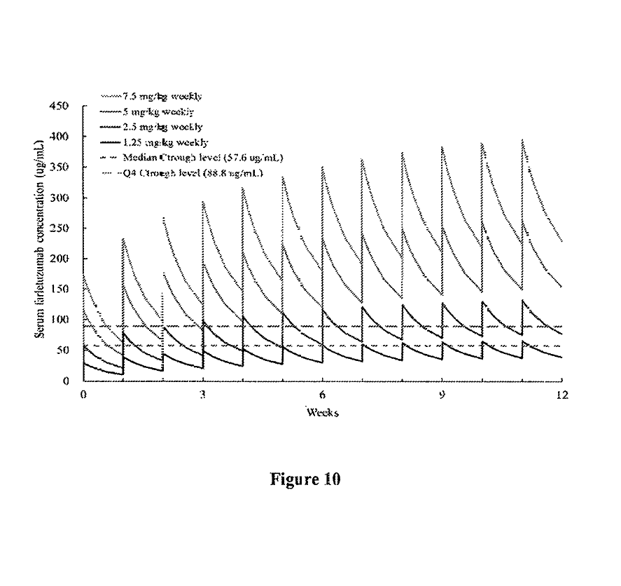

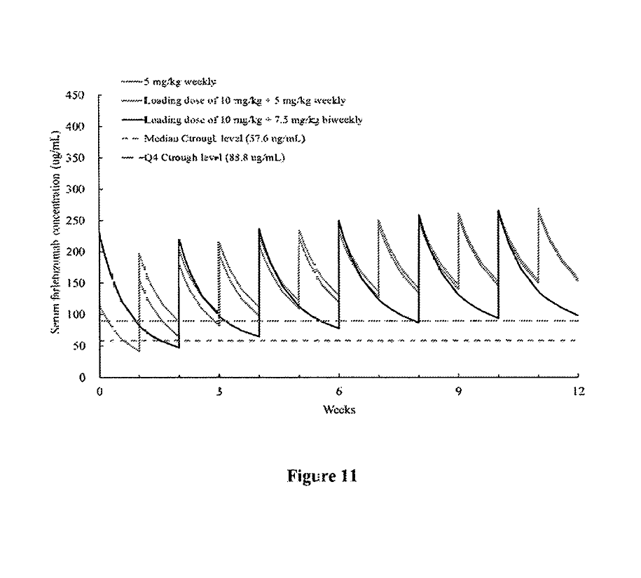

In addition, dose modeling simulations have been completed to illustrate several dose modifications including use of an initial loading dose and higher overall weekly doses that could assure patients obtain adequate minimum antibody concentration levels necessary for the intended treatment effect. FIG. 10 illustrates simulated weekly farletuzumab concentration-time profiles following administration of farletuzumab. Modeling has been used to compare farletuzumab concentration levels based on increasing weekly doses. Results of the exposure PFS analysis indicate that a median farletuzumab Cmin (or Ctrough) level of 57.6 μg/mL can correlate with an improvement of PFS, which is indicated in the lower dotted horizontal line. Weekly doses of 2.5 mg/kg have a 71% attainment rate to reach the median Ctrough level and a 28% attainment rate to reach a higher Q4 Ctrough level. The model indicates that a minimum dose of 5 mg/kg weekly is necessary to reach a 99% attainment rate for median Ctrough level and 89% attainment rate for the Q4 Ctrough target. FIG. 11 illustrates simulated farletuzumab concentration-time profiles following weekly and loading dose administration of farletuzumab. Modeling has been used to compare farletuzumab concentration levels based on higher weekly doses and an initial loading dose to reach target concentration levels faster. Results of the exposure PFS analysis indicate that a median Cmin (or Ctrough) level of 57.6 μg/mL correlates with an improvement of PFS, which is indicated in the lower dotted horizontal line. The model indicates that a minimum dose of 5 mg/kg farletuzumab weekly is necessary to reach a 99% attainment rate for median Ctrough, and the use of a 10 mg/kg farletuzumab loading dose demonstrates more rapid attainment of the target Ctrough level of both the median and Q4 level.

Other Evaluation

A mixed model using repeated measures ANOVA showed no treatment effect on QoL (FACT-O). Results for the functional domains (physical well-being, social/family well-being, emotional well-being, functional well-being, and ovarian cancer-specific modules) and composite measures (FACT-O and FACT-G) showed no differences due to treatment. Neither longitudinal analysis (Pattern Mixture Models and Generalized Estimating Equations) showed a statistically significant treatment effect.

Pharmacokinetics, Pharmacodynamics, Pharmacogenomics/Pharmacogenetics

Limited PK data were collected in the DDI substudy (N=7 subjects treated with farletuzumab or placebo+carboplatin/paclitaxel, N=0 subjects treated with farletuzumab or placebo+carboplatin/docetaxel). Mean plasma concentrations of free and total carboplatin and total paclitaxel concentration-time profiles were similar across all three treatment groups. As shown in Table 3, the total carboplatin PK and free carboplatin PK and total paclitaxel PK were similar between the two farletuzumab groups and the placebo group for mean clearance (CL), half-life (t1/2), total exposure (AUC0-inf), peak plasma level (Cmax), and time to reach Cmax (tmax).

TABLE 3

| | Parameter | | | | | | |
|---|---|---|---|---|---|---|---|
| Dose | (unit) | N | Mean | SD | Median | Min | Max |
| 1.25 mg/kg | Clearance | 365 | 0.0090 | 0.0030 | 0.0087 | 0.0029 | 0.0308 |
| | (L/h) | | | | | | |
| | Volume of | 365 | 3.02 | 0.71 | 2.93 | 1.30 | 8.46 |
| | central | | | | | | |
| | compartment | | | | | | |
| | (L) | | | | | | |
| | Inter- | 365 | 0.0134 | 0.0028 | 0.0131 | 0.0044 | 0.0354 |
| | compartment | | | | | | |
| | clearance | | | | | | |
| | (L/h) | | | | | | |
| | Volume of | 365 | 5.44 | 4.51 | 4.24 | 0.34 | 35.95 |
| | peripheral | | | | | | |
| | compartment | | | | | | |
| | (L) | | | | | | |
| | t_{1/2} of the | 365 | 700.5 | 311.7 | 637.5 | 253.3 | 3063.1 |
| | terminal | | | | | | |
| | phase (h) | | | | | | |
| | AUC | 365 | 10663.8 | 3883.3 | 10128.0 | 3203.1 | 28099.0 |
| | (μg · h/mL) | | | | | | |
| 2.5 mg/kg | Clearance | 364 | 0.0082 | 0.0027 | 0.0079 | 0.0028 | 0.0249 |
| | (L/h) | | | | | | |
| | Volume of | 364 | 2.93 | 0.61 | 2.87 | 0.53 | 5.24 |
| | central | | | | | | |
| | compartment | | | | | | |
| | (L) | | | | | | |
| | Inter- | 364 | 0.0136 | 0.0029 | 0.0133 | 0.0044 | 0.0297 |
| | compartment | | | | | | |
| | clearance | | | | | | |
| | (L/h) | | | | | | |
| | Volume of | 364 | 5.21 | 5.07 | 3.79 | 0.48 | 57.95 |
| | peripheral | | | | | | |
| | compartment | | | | | | |
| | (L) | | | | | | |
| | t_{1/2} of the | 364 | 720.3 | 351.0 | 638.9 | 192.6 | 4319.0 |
| | terminal | | | | | | |
| | phase (h) | | | | | | |
| | AUC | 364 | 22877.7 | 7063.9 | 22121.5 | 6639.3 | 51346.0 |
| | (μg · h/mL) | | | | | | |

1. A method for identifying a subject having a folate receptor alpha (FRA)-expressing ovarian cancer that will be responsive to treatment with an anti-FRA therapeutic agent, said method comprising determining a baseline level of cancer antigen 125 (CA125) of said subject; wherein a baseline CA125 level that is less than about eight times the upper limit of normal (ULN) for CA125 is indicative of a subject who would benefit from treatment with an anti-FRA therapeutic agent.

2. The method of claim 1 wherein a baseline CA125 level that is less than about three times the ULN for CA125 is indicative of a subject who would benefit from treatment with an anti-FRA therapeutic agent.

3. A method of treating a subject with folate receptor alpha (FRA)-expressing ovarian cancer, said method comprising:
determining a baseline level of cancer antigen 125 (CA125) of said subject, and
administering an anti-FRA therapeutic agent to said subject when said CA125 level is less than about eight times the upper limit of normal (ULN) for CA125.

4. The method of claim 3 wherein said anti-FRA therapeutic agent is administered to said subject when said baseline CA125 level in said biological sample is less than about three times the ULN for CA125.

5. The method of claim 3 wherein said step of determining a baseline level of CA125 of said subject is performed in vivo.

6. The method of claim 3 wherein said step of determining a baseline level of CA125 of said subject is performed on a biological sample obtained from said subject.

7. The method of claim 6 wherein said step of determining a baseline level of CA125 of said subject comprises contacting said biological sample with an anti-CA125 antibody.

8. The method of claim 6 wherein said biological sample used in determining said baseline level of CA125 comprises whole blood, serum, plasma, pleural effusion, ascites, or a tissue.

9. The method of claim 3, wherein said step of determining a baseline level of CA125 comprises using an antibody to detect protein expression, nucleic acid hybridization, quantitative RT-PCR, western blot analysis, radioimmunoassay, immunofluorimetry, immunoprecipitation, equilibrium dialysis, immunodiffusion, electrochemiluminescence (ECL) immunoassay, immunohistochemistry, fluorescence-activated cell sorting (FACS), or ELISA assay.

10. The method of claim 3 wherein said anti-FRA therapeutic agent comprises farletuzumab.

11. The method of claim 3 wherein said ovarian cancer is epithelial ovarian cancer.

12. The method of claim 3 wherein said ovarian cancer is platinum-sensitive.

13. The method of claim 3 wherein said ovarian cancer is platinum-resistant.

14. The method of claim 3 further comprising determining a baseline level of albumin of said subject, wherein a baseline serum albumin (S) concentration of at least 3.2 g/dL is further indicative of a subject who would benefit from treatment with an anti-FRA therapeutic agent.

15. The method of claim 14 wherein said baseline SA concentration is determined ex vivo or in vivo.

16. The method of claim 3 further comprising determining the level of folate receptor alpha (FRA) of in a sample derived from said subject by contacting said sample with an antibody that binds FRA and comparing the level of FRA in said sample derived from said subject with the level of FRA in a control sample, wherein an increase in the level of FRA in the sample derived from said subject as compared to the level of FRA in the control sample is indicative that the subject would benefit from treatment with an anti-FRA therapeutic agent.

17. The method according to claim 16 wherein the sample derived from said subject for determining the level of FRA is a tumor biopsy, urine, serum, plasma, or ascites.

18. The method according to claim 16 wherein the antibody that binds FRA is:
(a) an antibody that binds the same epitope as the MORAb-003 antibody;
(b) an antibody comprising SEQ ID NO: 1 (GFTFSGYGLS) as CDRH1, SEQ ID NO: 2 (MISSGGSYTYYADSVKG) as CDRH2, SEQ ID NO: 3 (HGDDPAWFAY) as CDRH3, SEQ ID NO:4 (SVSSSISSNNLH) as CDRL1, SEQ ID NO: 5 (GTSNLAS) as CDRL2 and SEQ ID NO: 6 (QQWSSYPYMYT) as CDRL3;
(c) the 548908 antibody;
(d) an antibody that binds the same epitope as the 548908 antibody;
(e) the 6D398 antibody;
(f) an antibody that binds the same epitope as the 6D398 antibody;
(g) an antibody that binds the same epitope as the 26B3 antibody;
(h) an antibody comprising SEQ ID NO: 14 (GYFMN) as CDRH1, SEQ ID NO: 15 (RIFPYNGDTFYNQKFKG) as CDRH2, SEQ ID NO: 16 (GTHYFDY) as CDRH3, SEQ ID NO: 17 (RTSENIFSYLA) as CDRL1, SEQ ID NO:18 (NAKTLAE) as CDRL2 and SEQ ID NO: 19 (QHHYAFPWT) as CDRL3;
(i) the 26B3 antibody;
(j) an antibody that binds the same epitope as the 19D4 antibody;
(k) an antibody comprising SEQ ID NO: 20 (HPYMH) as CDRH1, SEQ ID NO: 21 (RIDPANGNTKYDPKFQG) as CDRH2, SEQ ID NO: 22 (EEVADYTMDY) as CDRH3, SEQ ID NO: 23 (RASESVDTYGNNFIH) as CDRL1, SEQ ID NO: 24 (LASNLES) as CDRL2 and SEQ ID NO:25 (QQNNGDPWT) as CDRL3;
(l) the 19D4 antibody;
(m) an antibody that binds the same epitope as the 9F3 antibody;
(n) an antibody comprising SEQ ID NO:26 (SGYYWN) as CDRH1, SEQ ID NO:27 (YIKSDGSNNYNPSLKN) as CDRH2, SEQ ID NO:28 (EWKAMDY) as CDRH3, SEQ ID NO:29 (RASSTVSYSYLH) as CDRL1, SEQ ID NO:30 (GTSNLAS) as CDRL2 and SEQ ID NO:31 (QQYSGYPLT) as CDRL3;
(o) the 9F3 antibody;
(p) an antibody that binds the same epitope as the 24F12 antibody;
(q) an antibody comprising SEQ ID NO:32 (SYAMS) as CDRH1, SEQ ID NO:33 (EIGSGGSYTYYPDTVTG) as CDRH2, SEQ ID NO:34 (ETTAGYFDY) as CDRH3, SEQ ID NO:35 (SASQGINNFLN) as CDRL1, SEQ ID NO:36 (YTSSLHS) as CDRL2 and SEQ ID NO:37 (QHFSKLPWT) as CDRL3;
(r) the 24F12 antibody;
(s) an antibody that comprises a variable region light chain selected from the group consisting of LK26HuVK (SEQ ID NO: 38); LK26HuVKY (SEQ ID NO: 39); LK26HuVKPW (SEQ ID NO: 40); and LK26HuVKPW,Y (SEQ ID NO: 41);
(t) an antibody that comprises a variable region heavy chain selected from the group consisting of LK26HuVH (SEQ ID NO: 42); LK26HuVH FAIS,N (SEQ ID NO: 43); LK26HuVHSLF (SEQ ID NO: 44); LK26HuVH 1,1 (SEQ ID NO: 45); and LK26KOLHuVH (SEQ ID NO: 46);
(u) an antibody that comprises the heavy chain variable region LK26KOLHuVH (SEQ ID NO: 46) and the light chain variable region LK26HuVKPW,Y (SEQ ID NO: 41);
(v) an antibody that comprises the heavy chain variable region LK26HuVH SLF (SEQ ID NO: 44) and the light chain variable region LK26HuVKPW,Y (SEQ ID NO: 41);
(w) an antibody that comprises the heavy chain variable region LK26KOLHuVH (SEQ ID NO: 46) and the light chain variable region LK26HuVKPW,Y (SEQ ID NO: 41); and
(x) an antibody that comprises the heavy chain variable region LK26HuVH FAIS,N (SEQ ID NO: 43) and the light chain variable region LK26HuVKPW,Y (SEQ ID NO: 41).

19. The method of claim 16, wherein the antibody that binds FRA is labeled.

20. The method of claim 19, wherein the antibody that binds FRA is labeled with a radiolabel, a biotin-label, a chromophore-label, a fluorophore-label, an ECL label, or an enzyme-label.

21. The method of claim 16, wherein the level of FRA is determined by using a sandwich assay, western blot analysis, radioimmunoassay, immunofluorimetry, immunoprecipitation, equilibrium dialysis, immunodiffusion, solution phase assay, electrochemiluminescence immunoassay (ECLIA), or an ELISA assay.

22. The method of claim 16, wherein the control sample comprises a standardized control level of FRA in a healthy subject.

23. The method of claim 3 wherein said anti-FRA therapeutic agent is farletuzumab and wherein farletuzumab is administered to achieve a minimum serum farletuzumab concentration of at least about 57.6 μg/ml.

24. The method of claim 3 wherein said anti-FRA therapeutic agent is farletuzumab and wherein farletuzumab is administered to achieve a minimum serum farletuzumab concentration of at least about 88.8 μg/ml.

25. The method of claim 3 wherein said anti-FRA therapeutic agent is farletuzumab, wherein serum farletuzumab concentration in said subject is determined, and wherein a minimum serum farletuzumab concentration of at least about 57.6 μg/ml is indicative of a positive therapeutic response for said subject.

26. The method of claim 3 wherein said anti-FRA therapeutic agent is farletuzumab, wherein serum farletuzumab concentration in said subject is determined, and wherein a minimum serum farletuzumab concentration of at least about 88.8 μg/ml is indicative of a positive therapeutic response for said subject.

27. The method of claim 3 wherein said anti-FRA therapeutic agent is farletuzumab, wherein the average farletuzumab area under the curve pharmacokinetic exposure level is determined, and wherein average farletuzumab area under the curve pharmacokinetic exposure level of at least about 15.22 mg·h/L is indicative of a positive therapeutic response for said subject.

28. The method of claim 3 wherein said anti-FRA therapeutic agent is farletuzumab, wherein the average farletuzumab area under the curve pharmacokinetic exposure level is determined, and wherein average farletuzumab area under the curve pharmacokinetic exposure level of at least about 22.2 mg·h/L is indicative of a positive therapeutic response for said subject.

29. The method of claim 3 wherein said step of administering comprises intravenous injection of said anti-FRA therapeutic agent.

30. The method of claim 3 wherein said step of administering comprises intraperitoneal administration of said anti-FRA therapeutic agent.

31. The method of claim 3 wherein said step of administering comprises weekly administration of said anti-FRA therapeutic agent to said subject.

32. The method of claim 3 wherein said anti-FRA therapeutic agent is administered at a dose of about 2.5 mg/kg to about 10 mg/kg.

33. The method of claim 3 wherein said anti-FRA therapeutic agent is administered at a dose of about 5.0 mg/kg to about 7.5 mg/kg.

34. The method of claim 3 wherein said step of administering comprises administering a loading dose of said anti-FRA therapeutic agent of about 7.5 mg/kg to about 12.5 mg/kg to said subject.

35. The method of claim 34 wherein said step of administering further comprises administering a second loading dose of said anti-FRA therapeutic agent of about 7.5 mg/kg to about 12.5 mg/kg to said subject.

36. The method of claim 34 wherein said loading dose is about 10 mg/kg.

37. The method of claim 3 further comprising administering a platinum-containing compound to said subject.

38. The method of claim 3 further comprising administering a taxane to said subject.

39. The method of claim 37 further comprising administering a taxane to said subject.

40. The method of claim 37 wherein said platinum-containing compound comprises cisplatin or carboplatin.

41. The method of claim 38 wherein said taxane comprises paclitaxel, docetaxel, nab-paclitaxel, cabazitaxel, DJ-927, paclitaxel poliglumex, XRP9881, EndoTAG+paclitaxel, Polymeric-micellar paclitaxel, DHA-paclitaxel, and BMS-184476.

42. The method of claim 37 wherein said platinum-containing compound is administered once every three weeks.

43. The method claim 38 wherein said taxane is administered once every three weeks.

44. The method of claim 39 wherein said taxane is administered before, after, or simultaneously with said platinum-containing compound.

45. The method of claim 3 wherein said subject received surgical resection of the ovarian cancer, first-line platinum-based therapy, first-line taxane-based therapy, and/or first-line platinum and taxane-based therapy for treatment of the ovarian cancer for treatment of said ovarian cancer prior to said step of determining said baseline level of CA125.

46. The method of claim 45 wherein said subject exhibits symptomatic progression, serologic progression, and/or radiologic progression of said ovarian cancer prior to said step of determining said baseline level of CA125.

47. The method of claim 3 wherein said step of determining said baseline level of CA125 comprises determining a CA125 level in said subject at a single timepoint.

48. The method of claim 3 wherein said step of determining said baseline level of CA125 comprises determining a CA125 level in said subject at at least two timepoints.

49. The method of claim 3 wherein said subject received first-line platinum-based therapy.

50. A kit for identifying a subject having ovarian cancer that will be responsive to treatment with an anti-folate receptor alpha (FRA) therapeutic agent comprising an anti-CA125 antibody, a vessel for containing the antibody when not in use, and instructions for using said anti-CA125 antibody for determining a baseline level of CA125 in a biological sample obtained from said subject.

51. (canceled)

52. (canceled)

53. The kit of claim 50, wherein said anti-FRA therapeutic agent comprises farletuzumab.

54. The kit of claim 50 further comprising an anti-folate receptor alpha (FRA) antibody, a vessel for containing the anti-FRA antibody when not in use, and instructions for using said anti-FRA antibody for determining a level of FRA in a biological sample obtained from said subject.

55. The kit of claim 50 further comprising an anti-serum albumin (SA) antibody, a vessel for containing the anti-SA antibody when not in use, and instructions for using said anti-SA antibody for determining a level of SA in a biological sample obtained from said subject.

56. A kit for treating a subject having ovarian cancer that will be responsive to treatment with an anti-folate receptor alpha (FRA) therapeutic agent comprising said anti-FRA therapeutic agent, a vessel for containing said anti-FRA therapeutic agent when not in use, and instructions for use of said anti-FRA therapeutic agent, wherein said instructions specify that a baseline CA125 level that is less than about eight times the upper limit of normal for CA125 is indicative of a subject who would benefit from treatment with said anti-FRA therapeutic agent.

57. (canceled)

58. The kit of claim 56 further comprising an anti-CA125 antibody, a vessel for containing said antibody when not in use, and instructions for using said anti-CA125 antibody for determining a baseline level of CA125 of said subject.

59. The kit of claim 56 wherein said anti-FRA therapeutic agent comprises farletuzumab.

60. The kit of claim 56 further comprising an anti-folate receptor alpha (FRA) antibody, a vessel for containing the anti-FRA antibody when not in use, and instructions for using said anti-FRA antibody for determining a level of FRA in a biological sample obtained from said subject.

61. The kit of claim 56 further comprising an anti-serum albumin (SA) antibody, a vessel for containing the anti-SA antibody when not in use, and instructions for using said anti-SA antibody for determining a level of SA in a biological sample obtained from said subject.

62. The method of claim 14 further comprising determining the level of folate receptor alpha (FRA) in a sample derived from said subject by contacting said sample with an antibody that binds FRA and comparing the level of FRA in said sample derived from said subject with the level of FRA in a control sample, wherein an increase in the level of FRA in the sample derived from said subject as compared to the level of FRA in the control sample is indicative that the subject would benefit from treatment with an anti-FRA therapeutic agent.

63. (canceled)

64. The kit of claim 58 further comprising an anti-folate receptor alpha (FRA) antibody, a vessel for containing the anti-FRA antibody when not in use, and instructions for using said anti-FRA antibody for determining a level of FRA in a biological sample obtained from said subject.

65. (canceled)

66. The kit of claim 64 further comprising an anti-serum albumin (SA) antibody, a vessel for containing the anti-SA antibody when not in use, and instructions for using said anti-SA antibody for determining a level of SA in a biological sample obtained from said subject.

67. The kit of claim 54 further comprising an anti-serum albumin (SA) antibody, a vessel for containing the anti-SA antibody when not in use, and instructions for using said anti-SA antibody for determining a level of SA in a biological sample obtained from said subject.


SEQUENCE LISTING (8 sequences)

SEQ ID NO: 1 - protein, 10 aa, Artificial Sequence
  Description of Artificial Sequence Synthetic peptide
GFTFSGYGLS

SEQ ID NO: 2 - protein, 17 aa, Artificial Sequence
  Description of Artificial Sequence Synthetic peptide
MISSGGSYTYYADSVKG

SEQ ID NO: 3 - protein, 10 aa, Artificial Sequence
  Description of Artificial Sequence Synthetic peptide
HGDDPAWFAY

SEQ ID NO: 4 - protein, 12 aa, Artificial Sequence
  Description of Artificial Sequence Synthetic peptide
SVSSSISSNNLH

SEQ ID NO: 5 - protein, 7 aa, Artificial Sequence
  Description of Artificial Sequence Synthetic peptide
GTSNLAS

SEQ ID NO: 6 - protein, 11 aa, Artificial Sequence
  Description of Artificial Sequence Synthetic peptide
QQWSSYPYMYT

SEQ ID NO: 7 - protein, 217 aa, Artificial Sequence
  Description of Artificial Sequence Synthetic polypeptide
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 449 aa, Artificial Sequence
  Description of Artificial Sequence Synthetic polypeptide
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK